(19) United States
(12) Patent Application Publication
(10) Pub. No.: US 2024/0168011 A1
(43) Pub. Date: May 23, 2024

(54) CLEARANCE ASSAY

(71) Applicant: Akershus Universitetssykehus HF, Lørenskog (NO)

(72) Inventors: Tormod FLADBY, Blommenholm (NO); Marianne WETTERGREEN, Lillestrøm (NO); Kulbhushan SHARMA, Lørenskog (NO); Berglind GISLADOTTIR, Fjerdingby (NO); Bjørn-Eivind KIRSEBOM, Tomasjord (NO)

(73) Assignee: Akershus Universitetssykehus HF, Lørenskog (NO)

(21) Appl. No.: 18/549,388

(22) PCT Filed: Mar. 8, 2022
(86) PCT No.: PCT/EP2022/055950

§ 371 Date: Sep. 7, 2023

(30) Foreign Application Priority Data
Mar. 8, 2021 (EP) 21161384.9

(51) Int. Cl. G01N 33/50

(52) U.S. Cl. CPC G01N 33/5035; G01N 33/5055; G01N 2333/4709; G01N 2800/2821

(57) ABSTRACT

An ex vivo method of determining the effect of an agent on catalysis by a Monocyte Phagocyte System (MPS) cell of a disease marker product and/or at least one fragment thereof associated with neurodegeneration and/or inflammatory activation. The method comprises: i) maintaining a sample of MPS cells under conditions in which the MPS cells remain alive, ii) exposing the sample of MPS cells to an agent and a disease marker product, to permit phagocytosis of the disease marker product by the MPS cells, iii) detecting the intracellular amount of the disease marker product and/or at least one fragment thereof in the sample of MPS cells, and iv) comparing the intracellular amount of the disease marker product and/or the at least one fragment thereof to an intracellular amount of the same disease marker product and/or at least one fragment thereof measured in control MPS cells in the absence of the agent. The effect of the agent on catalysis by MPS cells of the disease marker product and/or at least one fragment thereof is determined by the result of the comparison of step iv).

### FIELD OF THE INVENTION

The present invention relates to a method of determining the effect of an agent on catalysis by a Monocyte Phagocyte System (MPS) cell of a disease marker product and/or at least one fragment thereof associated with neurodegeneration and/or inflammatory activation. The invention also relates to a kit for use in such a method.

### BACKGROUND

Aβ catabolism and clearance are essential at the synapse, in the brain parenchyma and along brain vasculature in order to avoid amyloid deposition in Alzheimer's Disease (AD) and cerebral amyloid angiopathies (CAA), respectively (Sikanyika, Parkington et al. 2019, Greenberg, Bacskai et al. 2020). Presynaptic and linked enzymatic activities instigate Aβ release to the perisynaptic interstitial fluid (ISF), from which diffusion, perivascular transport, microglial and neuronal uptake, aid clearance (Cirrito, Yamada et al. 2005, Roberts, Elbert et al. 2014, Bakker, Bacskai et al. 2016, Ries and Sastre 2016, Clayton, Van Enoo et al. 2017, Yuksel and Tacal 2019). Age, APOEE4 alleles, genetic factors linked to APP, Aβ, the presynaptic β-site APP cleaving enzyme 1 (BACE1) and myelogenic cells are the main risk factors for AD (Belloy, Napolioni et al. 2019, Jansen, Savage et al. 2019, Tamura, Chiu et al. 2020). Through clearance by diffusion, perivascular clearance and changes in neuronal and immune activity e.g. as part of diurnal patterns, daytime activities and intercurrent infections, these factors are kept in a homeostatic balance, thereby limiting formation of neurotoxic metabolites. This protective function is subserved by synaptic and parenchymal microglia, perivascular macrophages and pericytes. When homeostasis is sufficiently challenged, Aβ dysmetabolism, and deposition of amyloid plaques and perivascularamyloid follows. Brain deposition of other disease-related peptides and proteins is also linked to Monocyte Phagocytosis System (MPS) dysmetabolism in other neurodegenerative diseases and conditions associated with neurodegeneration. In AD and other neurodegenerative diseases, metabolic homeostasis may be challenged by genetic and acquired risk factors, inflammation, innate immune dysactivation and small vessel disease.

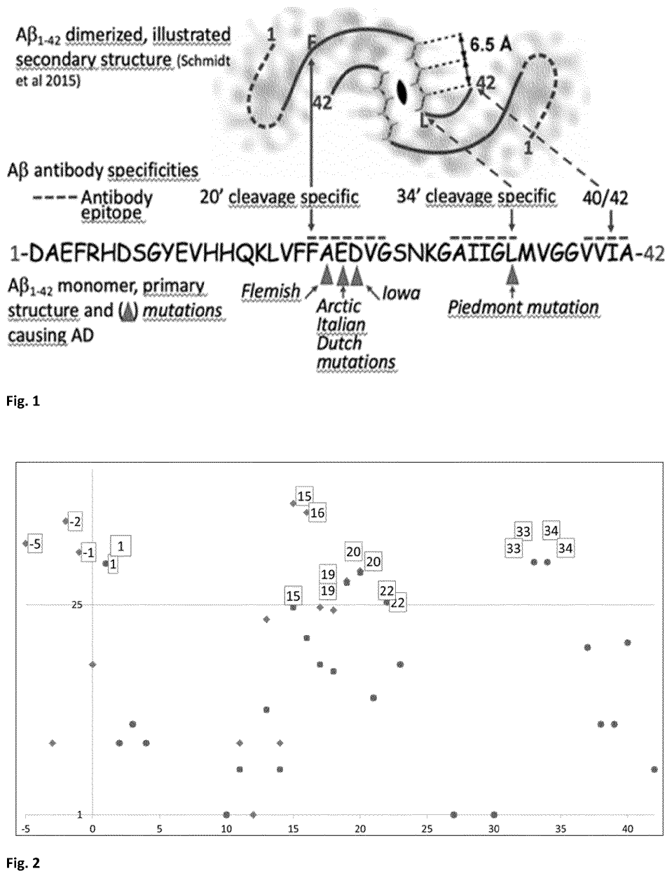

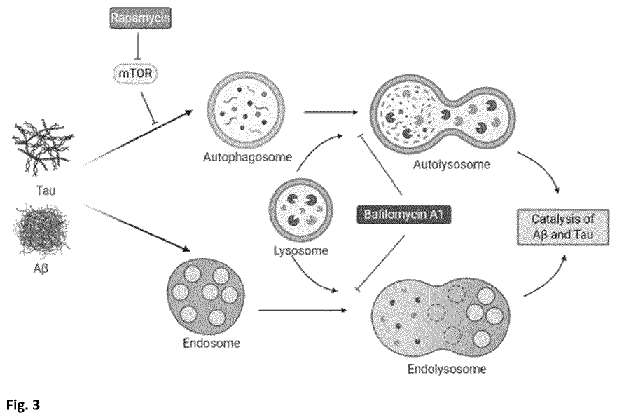

Clearance of Aβ is a complex process, of which phagocytosis and degradation/catabolism of AB are two important components. Catabolism in brain innate immune cells (i.e. microglia) and peripheral blood innate immune cells (e.g. monocytes and macrophages) contributes to Aβ clearance (Simard et al. 2006). Several proteases degrade Aβ both in vitro (Rogeberg et al. 2014) and in vivo (CTAD poster by Torsetnes et al. 2019), with intracellular, cell-membrane and extracellular activities. Protease activity may reflect cellular activation states, and could be manipulated for therapeutic purposes. Activity of intracellular proteases give rise to several Aβ mid-domain peptides (i.e. mid-domain fragments), with truncations at several different residues (Rogeberg et al. 2015). Two cleavage sites, between amino acid residue 19 (Phe) and 20 (Phe), and 34 (Leu) and 35 (Met), in the Aβ₁₋₄₂ nomenclature, generate the Aβ₂₀₋₃₄ peptide (FIGS. 1 and 2).

As toxicity is retained in shorter fragments of the Aβ peptide, which contain the central dimer/hairpin structure (FIG. 1), which also may inhibit proteolysis (Schmidt, Rohou et al. 2015). Supporting this, previous mass spectrometry experiments showed evidence for preferential intracellular Monocyte Phagocytosis System (MPS) catalysis of Aβ₁₋₄₂ at approximately amino acid residues 20 and 33/34 thereof, but a lack of catalysis in the intervening segment. These experiments also showed evidence for endogenous origin of APP-derived peptides, presumably originating from recycling of endosomes and APP degradation (FIG. 2; Malik, Maddison et al. 2019). Indeed, the mid-domain Aβₓ₋₃₄ fragment, which results from cleavage between amino acid residues 34 and 35 of Aβ₁₋₄₂, has been suggested to be a marker of amyloid clearance (Henium et al., 2020).

Therapeutically-enhanced Aβ catabolism is an attractive focus for AD treatment. Rapamycin analogues (“Rapalogs”), enhancement of endolysosomal enzyme activities and macroautophagy have been proposed for this purpose (Du, Liang et al. 2013, Jha, Jha et al. 2015, Malik, Maddison et al. 2019, Sikanyika, Parkington et al. 2019).

Inflammation also plays a role in neurodegenerative diseases such as AD, with neuroinflammation being regulated in part through the neuroimmune axis, where stimulation of α7-nicotinic receptors (α7 nicotinic acetylcholine receptors; α7 nAChR) on innate immune cells is an important component (Maldifassi et al., 2018; Eduardo et al., 2019). It has been shown that progression towards AD is also characterized by inflammation (Nordengen et al. 2019), and it is currently thought that microglia properties change in incipient AD, and acquire an inflammatory phenotype as the patient progresses towards AD-induced dementia. Wang et al. (2015) demonstrated that Abeta-40, a common form of Aβ, decreases the production of specialized proresolving mediators (SPMs), which play a key role in the resolution of inflammation, by peripheral blood mononuclear cells (PBMCs). Polyunsaturated fatty acids (PUFAs), including Ω3 fatty acids (Ω3-PUFA), modify innate immune activity as demonstrated previously using Docosahexaenoic acid (DHA; IUPAC name (4Z, 7Z, 10Z, 13Z, 16Z, 19Z)-4, 7, 10, 13, 16, 19-docosahexaenoic acid))-rich supplements, and this type of intervention has been shown to ameliorate AD-associated PBMC and microglia profiles, and to be associated with improvements in cognition (Wang et al. 2015; Antonietta et al. 2012). Effects of Ω3 PUFAs on the MPS are mainly attributed to the free fatty acid receptor 4 (FFAR4) (Im, 2016). Ω3 PUFAs can indirectly inhibit NF-κB, counteracting inflammation (Dyall, 2015; Cirpo et al., 2015). NF-κB, and α7 agonists, including positive allosteric modulators (PAMs), may regulate α7 nAChR expression. While activated α7 nAChRs are linked to extensive anti-inflammation and immune modulatory reactions with potential protective roles for a number of diseases including critical illness, upregulated CHRFAM7A transcription and expression is known to hinder CHRNA7 expression or α7 nAChR function, putatively promoting inflammatory activation and hindering α7 nicotinergic signaling (Ren et al., 2017; Can et al., 2019; de Lucas-Cerrillo et al., 2011; Marioli et al., 2019).

AD patients, patients with other neurodegenerative diseases and patients with inflammatory conditions have increased innate immune activation, possibly in part due to activation of the LPS/Toll-like receptor system (Zhan, Stamova & Sharp, 2018; Gambuzza et al., 2014).

It has been shown that DHA treatment of cells in an innate immune model system increases Aβ₁₋₄₂ phagocytosis and degradation, and that the increased phagocytosis is mediated by increased CHRNA7-expression at the plasma membrane (WO 2020/212627). Thus, a combination treatment using a FFAR4 agonist and an α7 nAChR agonist or PAM has been proposed for the treatment of AD (WO 2020/212627).

Personalized or precision medicine is a rapidly developing field. Several different mutations of Aβ are known to cause AD (some of which are shown in FIG. 1), with different patients having different mutations. Similarly, innate immune-related epigenetic changes, such as due to lifestyle factors, such as smoking and obesity, are believed to influence the risk of developing AD, and can affect different parts of the MPS. Different mutations to Aβ and different epigenetic changes will affect the efficacy of different endolysosomal enzymes, in MPS cells, to catabolise Aβ and fragments thereof, thereby affecting clearance of Aβ. It is believed that different therapeutic drugs target different parts of the MPS, and influence different endolysosomal enzymes (as shown in FIG. 3). There is, therefore, a need to provide personalized or precision medicine in view of individual causes of AD.

Other conditions associated with neurodegeneration and/or inflammatory activation such as amyloid angiopathy, viral infections, Parkinson's disease, Multiple Sclerosis, Frontotemporal Dementias and Amyotrophic Lateral Sclerosis, are also characterized by a malfunctioning of the MPS, resulting in abnormal clearance of peptides and the presence of a biomarker product or fragments thereof. For example, Tau, which is associated with each of AD, Parkinson's disease (PD), Progressive Supranuclear Palsy and Frontotemporal Dementias, is also catalyzed by the MPS (FIG. 3). It has been found that proteins associated with neurodegeneration and/or inflammatory activation have regions which are associated with aggregation or accumulation of the peptides and/or neurotoxicity. For example, mutagenesis has shown the importance of the R3 region of Tau (residues 306-336) in inducing aggregation of Tau, and a fragment corresponding to residues 244-372 of Tau reproduces much of the aggregation behavior of Tau in cells and animal models (Stohr et al., 2017). Two regions of TDP-43, namely residues 246-258 and 311-323, in the RRM-2 domain and C-terminal domain of TDP-43, respectively, have been found to show significant aggregation (Saini & Chauhan, 2011). Within these regions, residues 246-255 and 311-320 were found to be highly-aggregation prone (Saini & Chauhan, 2011). Cao et al (2019) reported that SegA (residues 311-360) and SegB (residues 286-331) of TDP-43 appear to be the pathogenic core of human TDP-43 aggregation. Myelin basic protein (MBP) also aggregates inside oligodendrocytes or around neuronal cells in demyelinating diseases, and it has previously been suggested that MBP binding may induce neuron-specific toxicity (Frid et al., 2015). It has also been suggested that MBP aggregates in a form which is resistant to degradation (Frid et al., 2015). In addition, selective MPS endolysosomal catalysis of viral capsids is in some cases required for viral reproduction, and is enhanced by the LPS/Toll-like receptor system (Pislar et al., 2020; Creasy & McCoy et al., 2011). Identical catalytic enzymes take part in catalysis of Aβ protein (Hook et al., 2002; Cermak et al., 2016). Thus, it is expected that catabolism of disease marker products associated with other neurodegeneration and/or inflammatory activation, which is associated with MPS malfunction, also results in catabolism products which are measurable and are related to essential elements of disease progression that may be targeted for intervention.

There is, therefore, a need to study the catabolism and intracellular clearance by MPS cells of disease marker products, and fragments thereof, of neurodegenerative diseases associated with a malfunctioning MPS. There is also a need to study the effect of inflammatory activation, particularly that associated with neurodegenerative diseases and MPS malfunctions, on intracellular clearance and the modulation thereof. There is also a need to characterize how different therapies modulate MPS functions and the clearance of disease marker products and fragments thereof, particularly in view of the different treatment targets within the MPS, and to identify new and improved therapies for treating neurodegenerative diseases and inflammatory activation. Furthermore, there is a need to develop personalized medicine methods in view of individual genetic and epigenetic variants associated with neurodegenerative diseases.

### SUMMARY OF THE INVENTION

The present invention arises from the finding that compromised CNS disease biomarker product homeostasis can be interrogated by measuring and monitoring catabolism by innate immune cells, i.e. the MPS, and that homeostasis can be ameliorated by modulating innate immune activation. In particular, the present invention arises from the finding that regions of proteins associated with aggregation and/or toxicity in neurodegenerative diseases and MPS malfunctions correspond to fragments which are resistant to catalysis. Thus, compromised CNS disease biomarker product homeostasis can be interrogated by measuring and monitoring catabolism of these mid-domain fragments.

Thus, in a first aspect of the invention, there is provided an ex vivo method of determining the effect of an agent on catalysis by a Monocyte Phagocyte System (MPS) cell of a disease marker product and/or at least one fragment thereof associated with neurodegeneration and/or inflammatory activation, wherein said method comprises:
i) maintaining a sample of MPS cells under conditions in which the MPS cells remain alive,
ii) exposing the sample of MPS cells to an agent and a disease marker product, to permit phagocytosis of the disease marker product by the MPS cells,
iii) detecting the intracellular amount of the disease marker product and/or at least one fragment thereof in the sample of MPS cells, and
iv) comparing the intracellular amount of the disease marker product and/or the at least one fragment thereof to an intracellular amount of the same disease marker product and/or at least one fragment thereof measured in control MPS cells in the absence of the agent,
   wherein the effect of the agent on catalysis by MPS cells of the disease marker product and/or at least one fragment thereof is determined by the result of the comparison of step iv).

Preferably, the MPS cells are microglial cells, macrophages or monocytes, preferably monocytes.

Conveniently, the method comprises the step of differentiating monocytes into macrophages.

Advantageously, the MPS cells are obtained from a human subject.

Preferably, the MPS cells are obtained from a CSF sample or a blood sample.

Conveniently, the MPS cells are obtained from a blood sample.

Advantageously, step ii) comprises culturing the MPS cells.

Conveniently, the agent is a stimulator or inhibitor of the MPS autophagic and/or endolysosomal systems, or is an inflammatory activator.

Preferably, the agent is Aβ₁₋₄₂, Aβ₁₋₄₀, a FFAR4 agonist, an α7 nAChR agonist or PAM, Rapamycin, Bafilomycin, Neprilysin inhibitor, IDE inhibitor, BACE inhibitor, a combination of a FFAR4 agonist and an α7 nAChR agonist or PAM, or a combination of IDE inhibitor, Neprilysin inhibitor and BACE inhibitor, preferably wherein the FFAR4 agonist is DHA, the α7 nAChR agonist or PAM is NS1738, and the combination of a FFAR4 agonist and an α7 nAChR agonist or PAM is combination of DHA and NS1738.

Conveniently, the disease marker product and/or at least one fragment thereof is associated with Alzheimer's disease.

Advantageously, the disease marker product is Aβ₁₋₄₂ (SEQ ID NO: 1), Aβ₁₋₄₀ (SEQ ID NO: 2), Tau protein, α-synuclein, myelin basic protein (MBP) or TDP-43.

Conveniently, the at least one fragment is a fragment of Aβ₁₋₄₂ (SEQ ID NO: 1), Aβ₁₋₄₀ (SEQ ID NO: 2), Tau protein, α-synuclein, myelin basic protein (MBP) or TDP-43, wherein the fragment of Aβ₁₋₄₂ (SEQ ID NO: 1) comprises residue 41 of Aβ₁₋₄₂ (SEQ ID NO: 1).

Advantageously, step iii) of the method comprises detecting the intracellular amount of at least one fragment of the disease marker product.

Conveniently, step iii) of the method comprises detecting the intracellular amount of more than one fragment of the disease marker product.

Advantageously, step iii) of the method comprises detecting the intracellular amount of the fragment of Aβ₁₋₄₀ (SEQ ID NO: 2) or the fragment of Aβ₁₋₄₂ (SEQ ID NO: 1) comprising residue 41 of Aβ₁₋₄₂ (SEQ ID NO: 1) by detecting a region of the fragment comprising at least 4 amino acids including residue 21, residue 34 or residue 40 of Aβ₁₋₄₂ (SEQ ID NO: 1) or Aβ₁₋₄₀ (SEQ ID NO: 2).

Preferably, step iii) of the method comprises detecting a fragment of Aβ₁₋₄₂ (SEQ ID NO: 1) or Aβ₁₋₄₀ (SEQ ID NO: 2) by detecting a region of the fragment comprising at least 4 amino acids comprising residues 21-24, residues 31-34 or residues 39-40 of Aβ₁₋₄₂ (SEQ ID NO: 1) or Aβ₁₋₄₀ (SEQ ID NO: 2).

Conveniently, step iii) of the method comprises detecting a fragment of Aβ₁₋₄₂ (SEQ ID NO: 1) or Aβ₁₋₄₀ (SEQ ID NO: 2) by detecting the N-terminal of SEQ ID NO: 6, the C-terminal of SEQ ID NO: 6, a region of the fragment comprising residues 21-29 of Aβ₁₋₄₂ (SEQ ID NO: 1) or Aβ₁₋₄₀ (SEQ ID NO: 2), and/or a region of the fragment comprising residues 34-40 or 34-41 of Aβ₁₋₄₂ (SEQ ID NO: 1) or Aβ₁₋₄₀ (SEQ ID NO: 2).

Advantageously, step iii) of the method comprises detecting a fragment of Aβ₁₋₄₂ (SEQ ID NO: 1) or Aβ₁₋₄₀ (SEQ ID NO: 2) by detecting the N-terminal of SEQ ID NO: 6, the C-terminal of SEQ ID NO: 6, a region of the fragment comprising residues 21-29 of Aβ₁₋₄₂ (SEQ ID NO: 1) or Aβ₁₋₄₀ (SEQ ID NO: 2), and/or a region of the fragment comprising residues 34-40 or 34-41 of Aβ₁₋₄₂ (SEQ ID NO: 1) or Aβ₁₋₄₀ (SEQ ID NO: 2).

Preferably, step iii) of the method comprises detecting the fragment of Aβ₁₋₄₂ (SEQ ID NO: 1) or Aβ₁₋₄₀ (SEQ ID NO: 2) by detecting more than one of the N-terminal or C-terminal of SEQ ID NO: 6 and the regions of Aβ₁₋₄₂ (SEQ ID NO: 1) or Aβ₁₋₄₀ (SEQ ID NO: 2), preferably by detecting
i) a region of the fragment comprising at least 4 amino acids comprising residues 21-24 and a region of the fragment comprising at least 4 amino acids comprising residues 31-34 or 39-40,
ii) the N-terminal of SEQ ID NO: 6 and a region of the fragment comprising residues 34-40 or 34-41 of Aβ₁₋₄₂ (SEQ ID NO: 1) or Aβ₁₋₄₀ (SEQ ID NO: 2), preferably residues 1-6 of SEQ ID NO: 6 and a region of the fragment comprising residues 34-40 or 34-41 of Aβ₁₋₄₂ (SEQ ID NO: 1) or Aβ₁₋₄₀ (SEQ ID NO: 2);
iii) the C-terminal of SEQ ID NO: 6 and a region of the fragment comprising residues 21-24, preferably 21-29, of Aβ₁₋₄₂ (SEQ ID NO: 1) or Aβ₁₋₄₀ (SEQ ID NO: 2); and/or
iv) a region of the fragment comprising residues 21-24, preferably residues 21-29, of Aβ₁₋₄₂ (SEQ ID NO: 1) or Aβ₁₋₄₀ (SEQ ID NO: 2) and a region of the fragment comprising residues 34-40 or 34-41 of Aβ₁₋₄₂ (SEQ ID NO: 1) or Aβ₁₋₄₀ (SEQ ID NO: 2).

Preferably, the at least one fragment is Aβ₁₋₄₂ (SEQ ID NO: 1) or Aβ₁₋₄₀ (SEQ ID NO: 2) which has been cleaved between amino acid residues 19 and 20 and/or between amino acid residues 34 and 35. Therefore, the fragments of Aβ₁₋₄₂ (SEQ ID NO: 1) or Aβ₁₋₄₀ (SEQ ID NO: 2) detected in the method of the invention comprise residue 20 as the N-terminal thereof and/or residue 34 as the C-terminal thereof.

Preferably, step iii) of the method comprises detecting fragments of Aβ₁₋₄₂ (SEQ ID NO: 1) or Aβ₁₋₄₀ (SEQ ID NO: 2) by detecting the N-terminal of SEQ ID NO: 6 and detecting the C-terminal of SEQ ID NO: 6.

Advantageously, step iii) of the method comprises detecting fragments of Aβ₁₋₄₂ (SEQ ID NO: 1) or Aβ₁₋₄₀ (SEQ ID NO: 2) by detecting the N-terminal of SEQ ID NO: 6, detecting the C-terminal of SEQ ID NO: 6 and detecting the C-terminal of SEQ ID NO: 1 or SEQ ID NO: 2.

Preferably, step iii) of the method comprises using antibodies to detect the fragments of Aβ₁₋₄₂ (SEQ ID NO: 1) or Aβ₁₋₄₀ (SEQ ID NO: 2).

Advantageously, step iii) of the method comprises using an antibody specific for SEQ ID NO: 3 or 7, and an antibody specific for the C-terminal of SEQ ID NO: 6 or specific for SEQ ID NO: 4, to detect the fragments of Aβ₁₋₄₂ (SEQ ID NO: 1) or Aβ₁₋₄₀ (SEQ ID NO: 2).

Conveniently, step iii) of the method comprises using an antibody specific for SEQ ID NO: 3 or 7, an antibody specific for the C-terminal of SEQ ID NO: 6 or specific for SEQ ID NO: 4, and an antibody specific for SEQ ID NO: 5, 8 or 9 to detect the fragments of Aβ₁₋₄₂ (SEQ ID NO: 1) or Aβ₁₋₄₀ (SEQ ID NO: 2).

Preferably, step iii) of the method comprises detecting the intracellular amount of at least one fragment of α-synuclein (SEQ ID NO: 10), Tau (SEQ ID NO: 14), TDP-43 (SEQ ID NO: 17) or MBP (SEQ ID NO: 20).

Advantageously, the fragment is:
Tau (SEQ ID NO: 14) which has been cleaved between amino acid residues 280 and 281 and/or between amino acid residues 322 and 232; or between amino acid residues 305 and 306 and/or amino acid residues 322 and 323;
α-synuclein (SEQ ID NO: 10) which has been cleaved between amino acid residues 38 and 39 and/or between amino residues 53 and 54; between amino acid residues 52 and 53 and/or amino acid residues 72 and 73; or between amino acid residues 75 and 76 and/or amino acid residues 90 and 91:
TDP-43 (SEQ ID NO: 17) which has been cleaved between amino acid residues 310 and 311 and/or between amino acid residues 334 and 335 or between amino acid residues 320 and 321 or amino acid residues 323 and 324 or amino acid residues 360 and 261; between amino acid residues 245 and 246 and/or between amino acid residues 255 and 256; or between amino acid residues 285 and 285 and/or amino acid residues 331 and 332; or
MBP (SEQ ID NO: 20) which has been cleaved between amino acid residues 29 and 30 and/or between residues 70 and 71.

Therefore, the fragments of α-synuclein (SEQ ID NO: 10) detected in the methods of the present invention comprise residue 39, 53 or 76 as the N-terminal thereof and/or residue 53, 72 or 90 as the C-terminal thereof. However, when the N-terminal is residue 53, the C-terminal is not residue 53; and when the N-terminal is residue 76, the C-terminal is neither residue 53 nor 72. The fragments of Tau detected in the method of the invention comprise residue 281 or 306 as the N-terminal thereof and/or residue 322 as the C-terminal thereof. The fragments of TDP-43 (SEQ ID NO: 17) detected in the methods of the invention comprise residue 311 as the N-terminal thereof and/or residue 320 or 334 as the C-terminal thereof, or comprise residue 246 as the N-terminal thereof and/or residue 255 as the C-terminal thereof. The fragments of MBP (SEQ ID NO: 20) detected in the methods of the invention comprise residue 30 as the N-terminal thereof and/or residue 70 as the C-terminal thereof.

Conveniently, the fragment is:
Tau (SEQ ID NO: 14) which has been cleaved between amino acid residues 280 and 281 and/or between amino acid residues 322 and 323;
α-synuclein (SEQ ID NO: 10) which has been cleaved between amino acid residues 38 and 39 and/or between amino acid residues 53 and 54;
TDP-43 (SEQ ID NO: 17) which has been cleaved between amino acid residues 310 and 311 and/or between amino acid residues 334 and 335; or
MBP (SEQ ID NO: 20) which has been cleaved between amino acid residues 29 and 30 and/or between residues 70 and 71.

Conveniently, in step ii), the agent is a stimulator or activator of the MPS autophagic and/or endolysosomal systems, and step ii) comprises exposing the sample of MPS cells to an inflammatory activator in addition to the agent.

Advantageously, the inflammatory activator is LPS.

Preferably, the control MPS cells are cells which have undergone steps i) and ii) of the method, except that the control MPS cells have not been exposed to the agent.

Conveniently, the method is for comparing the effects of a panel of agents on catalysis of the disease marker product and/or at least one fragment thereof, and identifying the agent with the greatest effect on catalysis of the disease marker product and/or at least one fragment thereof.

Advantageously, the method is for screening agents for use in the treatment of the neurodegeneration and/or inflammatory activation and/or a condition associated with neurodegeneration.

Conveniently, the method is for use in diagnosing neurodegeneration, and/or inflammatory activation and/or a condition associated with neurodegeneration.

Preferably, the method is for use in personalised medicine, for determining whether an agent is an effective treatment for a human subject, wherein step i) of the method comprises maintaining a sample of MPS cells obtained from a human subject under conditions in which the MPS cells remain alive, and wherein the effectiveness of the agent on the human subject is determined by the result of the comparison of step iv).

In a second aspect of the invention, there is provided a kit comprising:
one or more antibodies selected from an antibody specific for a region of a fragment of Aβ₁₋₄₂ (SEQ ID NO: 1) or Aβ₁₋₄₀ (SEQ ID NO: 2) comprising at least 4 amino acids comprising residue 21 of Aβ₁₋₄₂ (SEQ ID NO: 1) or Aβ₁₋₄₀ (SEQ ID NO: 2), an antibody specific for a fragment of Aβ₁₋₄₂ (SEQ ID NO: 1) or Aβ₁₋₄₀ (SEQ ID NO: 2) comprising at least 4 amino acids comprising residue 34 of Aβ₁₋₄₂ (SEQ ID NO: 1) or Aβ₁₋₄₀ (SEQ ID NO: 2) and an antibody specific for a fragment of Aβ₁₋₄₂ (SEQ ID NO: 1) or Aβ₁₋₄₀ (SEQ ID NO: 2) comprising at least 4 amino acids comprising residue 40 of Aβ₁₋₄₂ (SEQ ID NO: 1) or Aβ₁₋₄₀ (SEQ ID NO: 2);
one or more antibodies selected from an antibody specific for a region of a fragment of α-synuclein (SEQ ID NO: 10) comprising at least 4 amino acids comprising residue 39 of α-synuclein (SEQ ID NO: 10), an antibody specific for a region of a fragment of α-synuclein (SEQ ID NO: 10) comprising at least 4 amino acids comprising residue 53 of α-synuclein (SEQ ID NO: 10), an antibody specific for a region of a fragment of α-synuclein (SEQ ID NO: 10) comprising at least 4 amino acids comprising residue 72 of α-synuclein (SEQ ID NO: 10), an antibody specific for a region of a fragment of α-synuclein (SEQ ID NO: 10) comprising at least 4 amino acids comprising residue 76 of α-synuclein (SEQ ID NO: 10) and an antibody specific for a region of a fragment of α-synuclein (SEQ ID NO: 10) comprising at least 4 amino acids comprising residue 90 of α-synuclein (SEQ ID NO: 10);
one or more antibodies selected from an antibody specific for a region of a fragment of Tau (SEQ ID NO: 14) comprising at least 4 amino acids comprising residue 306 of Tau (SEQ ID NO: 14), an antibody specific for a region of a fragment of Tau (SEQ ID NO: 14) comprising at least 4 amino acids comprising residue 281 of Tau (SEQ ID NO: 14) and an antibody specific for a region of a fragment of Tau (SEQ ID NO: 14) comprising at least 4 amino acids comprising residue 322 of Tau (SEQ ID NO: 14);
one or more antibodies selected from an antibody specific for a region of a fragment of TDP-43 (SEQ ID NO: 17) comprising at least 4 amino acids comprising residue 311 of TDP-43 (SEQ ID NO: 17), an antibody specific for a region of a fragment of TDP-43 (SEQ ID NO: 17) comprising at least 4 amino acids comprising residue 334 of TDP-43 (SEQ ID NO: 17), an antibody specific for a region of a fragment of TDP-43 (SEQ ID NO: 17) comprising at least 4 amino acids comprising residue 246 of TDP-43 (SEQ ID NO: 17) and an antibody specific for a region of a fragment of TDP-43 (SEQ ID NO: 17) comprising at least 4 amino acids comprising residue 255 of TDP-43 (SEQ ID NO: 17); or
one or more antibodies selected from an antibody specific for a region of a fragment of MBP (SEQ ID NO: 20) comprising at least 4 amino acids comprising residue 30 of MBP (SEQ ID NO: 20) and an antibody specific for a region of a fragment of MBP (SEQ ID NO: 20) comprising at least 4 amino acids comprising residue 70 of MBP (SEQ ID NO: 20).

Preferably, the kit comprises:
an antibody specific for a region of a fragment of Aβ₁₋₄₂ (SEQ ID NO: 1) or Aβ₁₋₄₀ (SEQ ID NO: 2) comprising at least 4 amino acids comprising residue 21 of Aβ₁₋₄₂ (SEQ ID NO: 1) or Aβ₁₋₄₀ (SEQ ID NO: 2), an antibody specific for a fragment of Aβ₁₋₄₂ (SEQ ID NO: 1) or Aβ₁₋₄₀ (SEQ ID NO: 2) comprising at least 4 amino acids comprising residue 34 of Aβ₁₋₄₂ (SEQ ID NO: 1) or Aβ₁₋₄₀ (SEQ ID NO: 2) and an antibody specific for a fragment of Aβ₁₋₄₂ (SEQ ID NO: 1) or Aβ₁₋₄₀ (SEQ ID NO: 2) comprising at least 4 amino acids comprising residue 40 of Aβ₁₋₄₂ (SEQ ID NO: 1) or Aβ₁₋₄₀ (SEQ ID NO: 2);
an antibody specific for a region of a fragment of α-synuclein (SEQ ID NO: 10) comprising at least 4 amino acids comprising residue 39 of α-synuclein (SEQ ID NO: 10) and an antibody specific for a region of a fragment of α-synuclein (SEQ ID NO: 10) comprising at least 4 amino acids comprising residue 53 of α-synuclein (SEQ ID NO: 10);
an antibody specific for a region of a fragment of Tau (SEQ ID NO: 14) comprising at least 4 amino acids comprising residue 281 of Tau (SEQ ID NO: 14) and an antibody specific for a region of a fragment of Tau (SEQ ID NO: 14) comprising at least 4 amino acids comprising residue 322 of Tau (SEQ ID NO: 14);
an antibody specific for a region of a fragment of TDP-43 (SEQ ID NO: 17) comprising at least 4 amino acids comprising residue 311 of TDP-43 (SEQ ID NO: 17) and an antibody specific for a region of a fragment of TDP-43 (SEQ ID NO: 17) comprising at least 4 amino acids comprising residue 334 of TDP-43 (SEQ ID NO: 17); or
an antibody specific for a region of a fragment of MBP (SEQ ID NO: 20) comprising at least 4 amino acids comprising residue 30 of MBP (SEQ ID NO: 20) and an antibody specific for a region of a fragment of MBP (SEQ ID NO: 20) comprising at least 4 amino acids comprising residue 70 of MBP (SEQ ID NO: 20).

Advantageously, the kit comprises one or more antibodies selected from an antibody specific for SEQ ID NO: 3, an antibody specific for SEQ ID NO: 4, an antibody specific for SEQ ID NO: 5.

Conveniently an antibody specific for SEQ ID NO: 7, an antibody specific for SEQ ID NO: 8 and an antibody specific for SEQ ID NO: 9.

Advantageously, the kit comprises an antibody specific for SEQ ID NO: 3, an antibody specific for SEQ ID NO: 4 and an antibody specific for SEQ ID NO: 5.

Preferably, the kit comprises an antibody specific for SEQ ID NO: 5, an antibody specific for SEQ ID NO: 7, an antibody specific for SEQ ID NO: 8 and an antibody specific for SEQ ID NO: 9.

The term “disease marker product”, as used herein, refers to a molecule wherein an abnormal level of the molecule within the human body is correlated with a disease condition. In some embodiments, the disease marker product is a disease marker protein or polypeptide.

The term “Monocyte Phagocytosis System cells”, or “MPS cells”, as used herein, refers to innate immune cells involved in phagocytosis and degradation of foreign bodies.

These cells all belong to the myeloid lineage, having gone through similar ontogenetic differentiation processes resulting in common epigenetic modifications, differentiating to different subsets only late in development. MPS cells include, but are not limited to, monocytes, macrophages, microglia and different types of tissue-specific macrophages (e.g. alveolar and interstitial lung macrophages), and are closely related to other myelogenic cells e.g. like skin dendritic cells.

The term “mid-domain peptide”, as used herein, refers to peptides produced by cleavage of proteins which are associated with neurodegeneration and/or MPS malfunction, and/or the pathology, symptoms and/or effects thereof, to produce fragments which are associated with aggregation and which are resistant to catalysis. For example, one mid-domain peptide is produced by cleavage of Aβ₁₋₄₂ between amino acid residues 19 and 20 and/or between amino acid residues 34 and 35. The term “mid-domain fragment” can be used interchangeably with the term “mid-domain peptide”.

The term “MPS stimulator”, as used herein, refers to an agent which increases phagocytosis and/or catalysis by the MPS. This term includes agents which increase the activity of endolysosomal enzymes.

The term “MPS inhibitor”, as used herein, refers to an agent which decreases phagocytosis and/or catalysis by the MPS. This term includes agents which decrease the activity of endolysosomal enzymes.

The term “inflammatory activator”, as used herein, refers to an agent which induces an inflammatory response.

The term “FFAR4”, as used herein, refers to a free fatty acid receptor which is a member of the ‘rhodopsin-like’ G-protein couple receptor (GPCR) family, and which is activated selectively by long chain fatty acids. FFAR4 was previously known as GPR120. Further details thereof may be found in Free Fatty Acid Receptors, Springer, 2018, pp 33-56, which is incorporated herein by reference.

The term “α7 nAChR”, as used herein, refers to the nicotinic acetylcholine receptor made up of five identical α7 subunits.

The term “agonist”, as used herein, refers to a substance which binds to and directly activates a receptor. It includes both full agonists and partial agonists (i.e. agonists which have only partial efficacy compared to a full agonist).

The term “omega-3 fatty acid”, as used herein, refers to a n-3 polyunsaturated fatty acid characterised by the presence of a double bond three atoms away from the terminal methyl group.

The term “positive modulator”, as used herein, refers to a substance which indirectly increases the effects of a primary ligand on a target protein.

The term “positive allosteric modulator”, as used herein, refers to a substance which indirectly induces an increase to the effects of an agonist on a target protein without directly activating the protein, by binding to a site distinct from the orthosteric binding site.

The term “a pharmaceutically acceptable salt thereof”, as used herein, means a salt formed by allowing the free form compound to react with an acid or base. Examples of pharmaceutically acceptable salts include hydrohalogenic acid salts such as hydrofluorides, hydrochlorides, hydrobromides, and hydroiodides; inorganic acid salts such as hydrochlorides, nitrates, perchlorates, sulfates and phosphates; lower alkanesulfonic acid salts such as methanesulfonates, trifluoromethanesulfonates, and ethanesulfonates; arylsulfonic acid salts such as benzenesulfonates, and p-toluenesulfonates; organic acid salts such as acetates, malates, fumarates, succinates, citrates, ascorbates, tartrates, oxalates, and maleates; alkali metal salts such as sodium salts, potassium salts, and lithium salts; alkaline earth metal salts such as calcium salts and magnesium salts; metal salts such as aluminum salts and iron salts; inorganic salts such as ammonium salts; amine salts including organic salts such as t-octylamine salts, dibenzylamine salts, morpholine salts, glucosamine salts, phenylglycine alkyl ester salts, ethylenediamine salts, N-methylglucamine salts, guanidine salts, diethylamine salts, triethylamine salts, dicyclohexylamine salts, N,N′-dibenzylethylenediamine salts, chloroprocaine salts, procaine salts, diethanolamine salts, N-benzylphenethylamine salts, piperazine salts, tetramethylammonium salts, and tris(hydroxymethyl)aminomethane salts; and amino acid salts such as glycine salts, lysine salts, arginine salts, ornithine salts, glutamates, and aspartates.

The term “pharmaceutical composition”, as used herein, means a pharmaceutical preparation suitable for administration to an intended human or animal subject for therapeutic purposes. In some embodiments it comprises a pharmaceutically acceptable carrier or diluent in addition to the active ingredient.

BRIEF DESCRIPTION OF THE FIGURES

FIG. 1 shows the secondary structure of dimerised Aβ₁₋₄₂ (Schmidt et al. 2015), the primary structure of Aβ₁₋₄₂ (SEQ ID NO: 1), the epitopes (SEQ ID NOs: 2-4) against which antibodies were raised, and the locations of the most common mutations in Aβ₁₋₄₂ causing AD.

FIG. 2 shows the peptide bond cleavage pattern of Aβ peptides found in monocytes. The squares show peptide bond breaks (pbbs) for peptides within Aβ₁₋₄₂, while the diamonds are pbbs from peptides from the entire APP sequence. The x-axis denotes the peptide bond number and the y-axis denotes the number of times each peptide bond is broken. The numbers in the boxes emphasise the most frequent cleavage site.

FIG. 3 shows the intracellular catalysis pathway of Aβ and Tau.

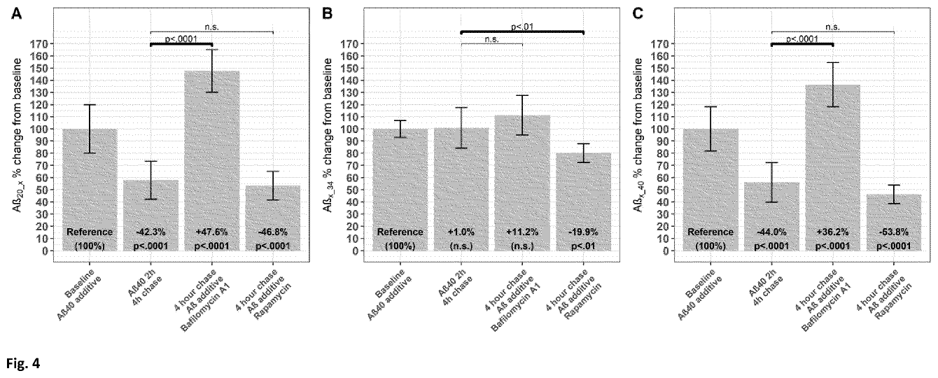

FIG. 4 shows the effect of an endolysosomal enzyme inhibitor (Bafilomycin) and an autophagosome activator (Rapamycin) on degradation of each Aβ₂₀₋ₓ(A), Aβₓ₋₃₄ (B) and Aβₓ₋₄₀ (C).

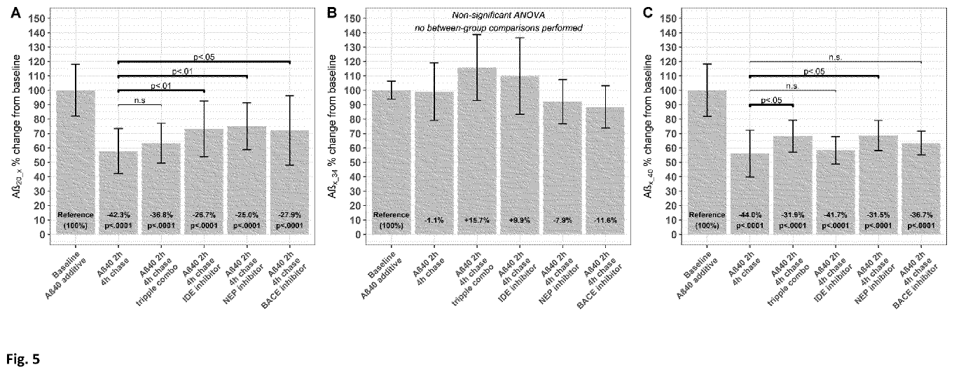

FIG. 5 shows the effect of different endolysosomal enzyme inhibitors (IDE inhibitor, Neprilysin inhibitor and BACE inhibtor), and a triple combination thereof, degradation of each Aβ₂₀₋ₓ (A), Aβₓ₋₃₄ (B) and Aβₓ₋₄₀ (C).

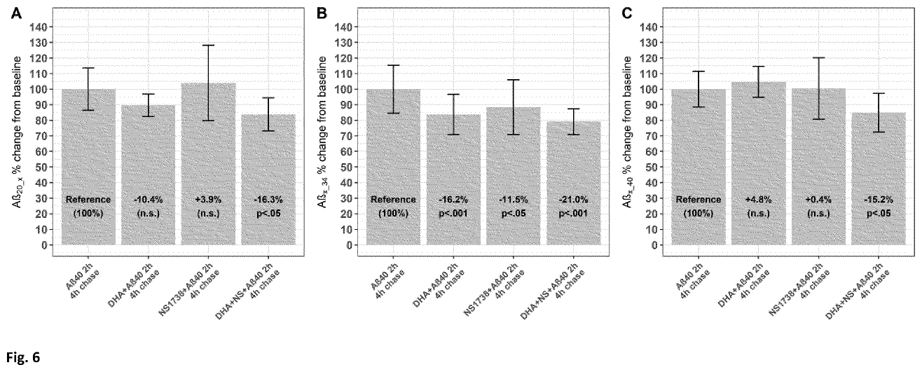

FIG. 6 shows the effect of each of a FFAR4 agonist (DHA), an α7 nAChR PAM (NS1738), and a combination of a FFAR4 agonist and an α7 nAChR PAM (DHA+NS1738) on degradation of each Aβ₂₀₋ₓ (A), Aβₓ₋₃₄ (B) and Aβₓ₋₄₀ (C).

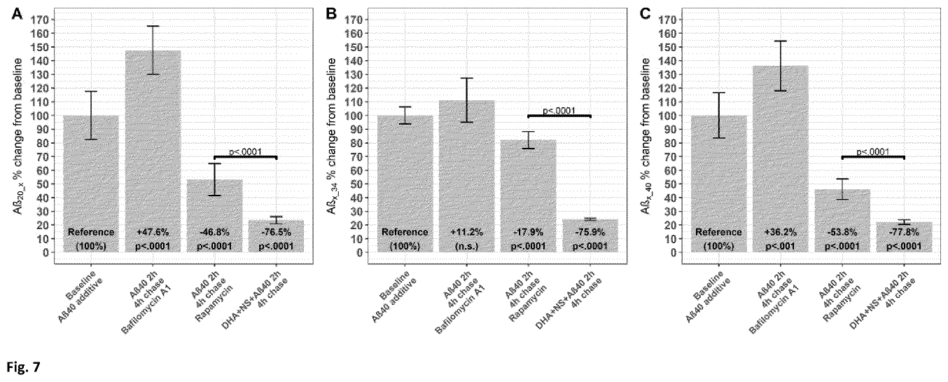

FIG. 7 shows the effect of combination of a FFAR4 agonist and an α7 nAChR PAM (DHA+NS1738) on degradation of each Aβ₂₀₋ₓ (A), Aβₓ₋₃₄ (B) and Aβₓ₋₄₀ (C) in comparison to the effect of a an endolysosomal enzyme inhibitor (Bafilomycin) and an autophagosome activator (Rapamycin).

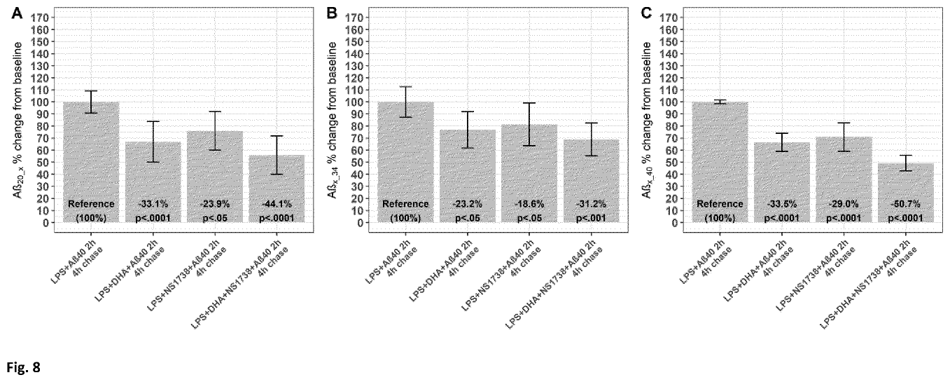

FIG. 8 shows effect of each of a FFAR4 agonist (DHA), an α7 nAChR PAM (NS1738), and a combination of a FFAR4 agonist and an α7 nAChR PAM (DHA+NS1738), on a background of inflammatory activation induced by LPS, on degradation of each Aβ₂₀₋ₓ (A), Aβₓ₋₃₄ (B) and Aβₓ₋₄₀ (C).

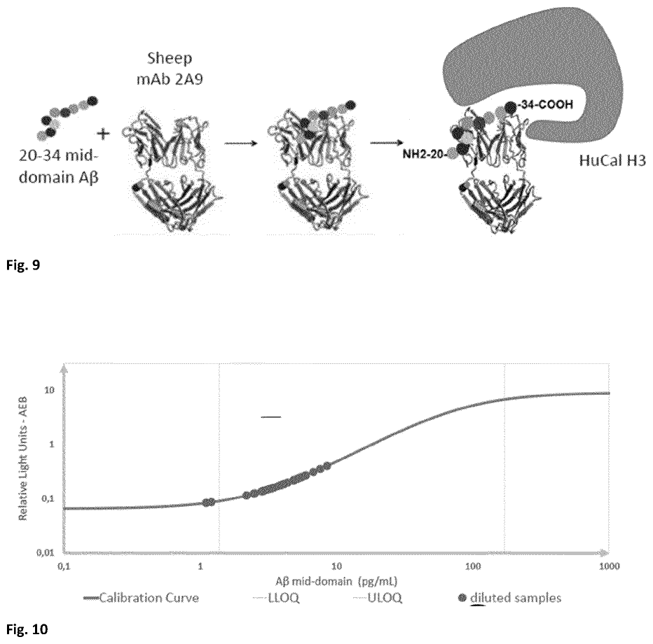

FIG. 9 shows the Aβ fragment and the antibodies used in the immunoassay of Example 4.

FIG. 10 shows the calibration curve obtained with monocyte samples in Example 4.

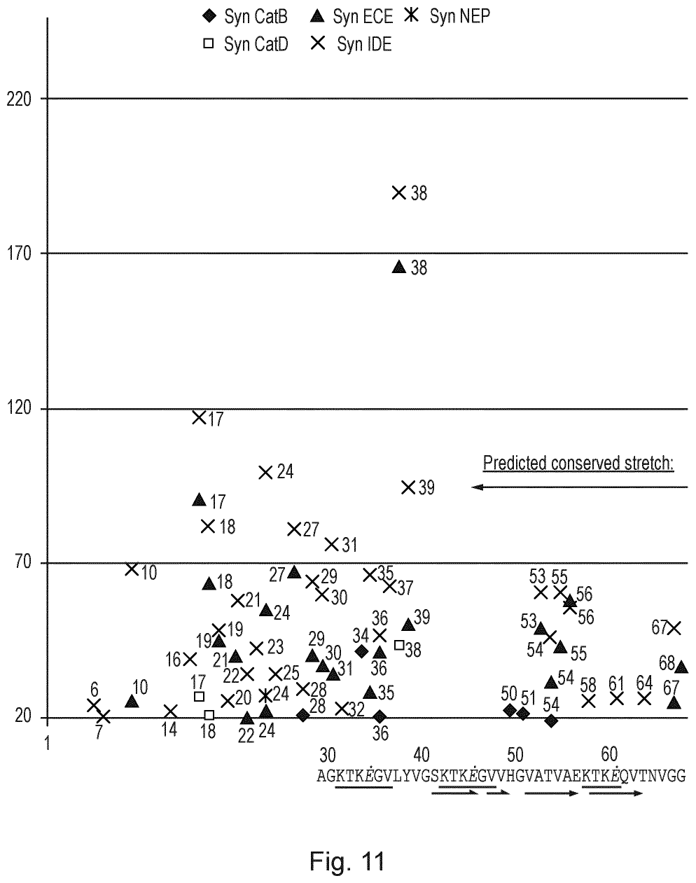

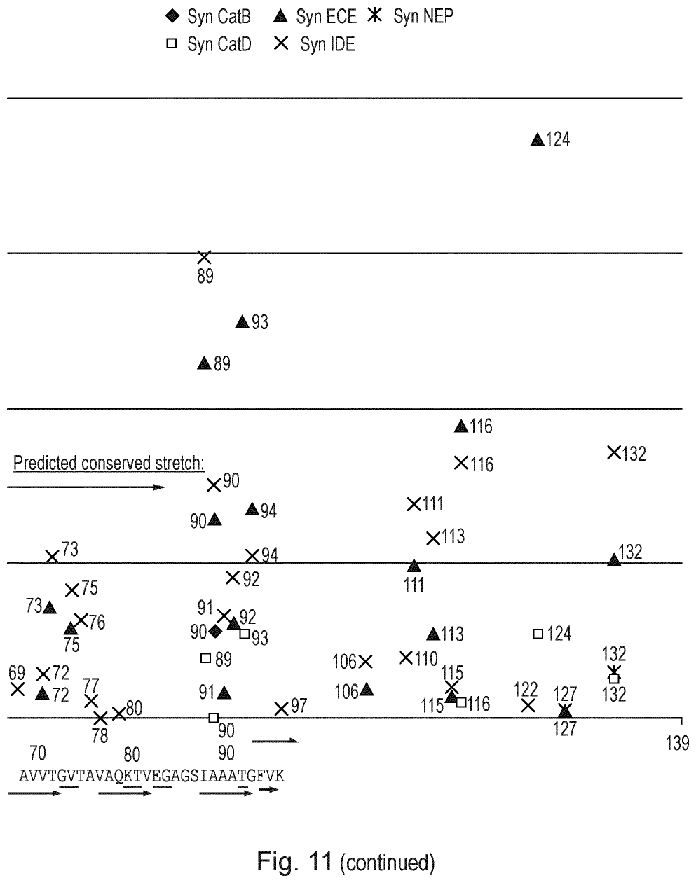

FIG. 11 shows the peptide bond cleavage pattern of α-synuclein by five different enzymes involved in intracellular and extracellular protein degradation: Cathepsin B (CatB), Cathepsin D (CatD), Endothelin Converting Enzyme-1 (ECE-1), Insulin degrading enzyme (IDE) and Neprilysin (NEP). The x-axis denotes the peptide bond number and the y-axis denotes the number of times each peptide bond is broken.

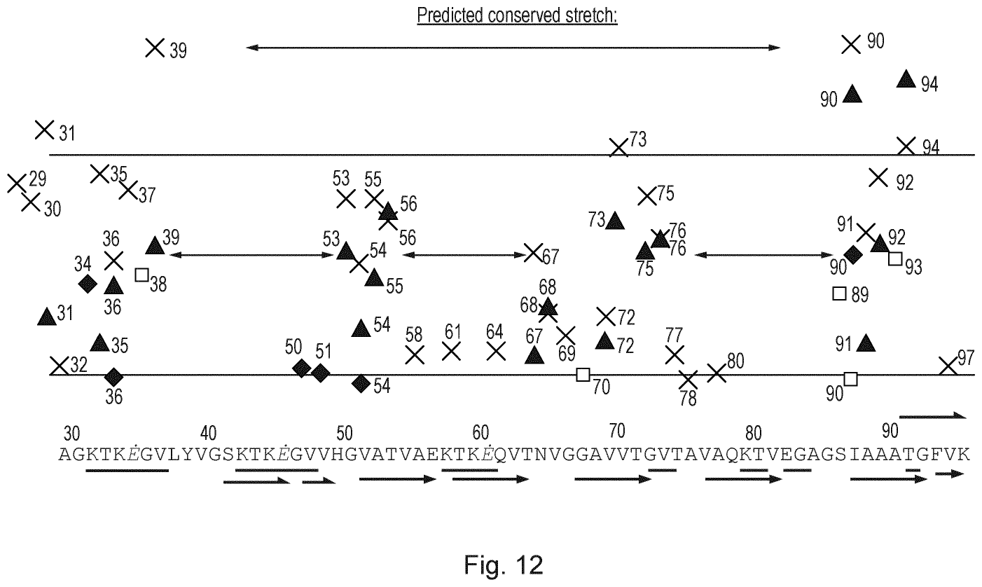

FIG. 12 shows an enlarged section of FIG. 11, showing that the predicted conserved stretch of FIG. 11 is in fact made up of three shorter conserved stretches.

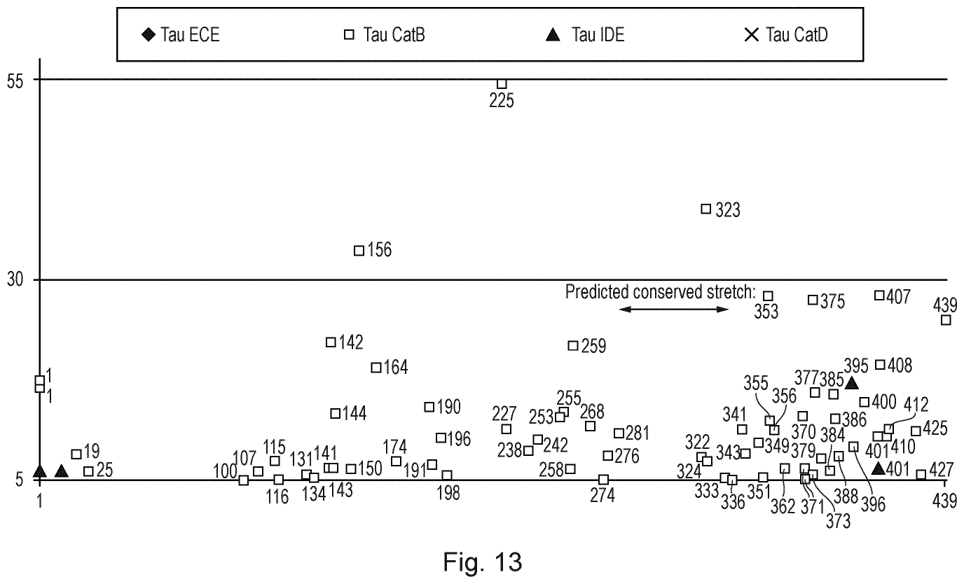

FIG. 13 shows the peptide bond cleavage pattern of Tau by four different enzymes involved in intracellular and extracellular protein degradation: Cathepsin B (CatB), Cathepsin D (CatD), Endothelin Converting Enzyme-1 (ECE-1) and Insulin degrading enzyme (IDE). The x-axis denotes the peptide bond number and the y-axis denotes the number of times each peptide bond is broken.

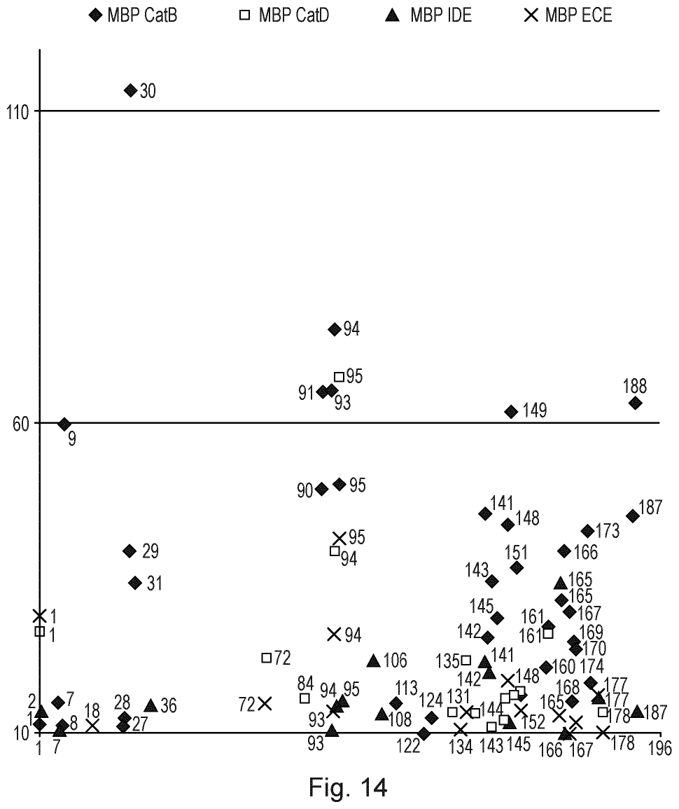

FIG. 14 shows the peptide bond cleavage pattern of MBP by four different enzymes involved in intracellular and extracellular protein degradation: Cathepsin B (CatB), Cathepsin D (CatD), Endothelin Converting Enzyme-1 (ECE-1) and Insulin degrading enzyme (IDE). The x-axis denotes the peptide bond number and the y-axis denotes the number of times each peptide bond is broken.

### BRIEF DESCRIPTION OF THE SEQUENCE LISTING

SEQ ID NO: 1 is the amino acid sequence of Aβ₁₋₄₂.

SEQ ID NO: 2 is the amino acid sequence of Aβ₁₋₄₀.

SEQ ID NO: 3 is the amino acid sequence of an epitope of Aβ₁₋₄₂ (SEQ ID NO: 1) and Aβ₁₋₄₀ (SEQ ID NO: 2) against which an antibody was raised.

SEQ ID NO: 4 is the amino acid sequence of an epitope of Aβ₁₋₄₂ (SEQ ID NO: 1) and Aβ₁₋₄₀ (SEQ ID NO: 2) against which an antibody was raised.

SEQ ID NO: 5 is the amino acid sequence of an epitope of Aβ₁₋₄₂ (SEQ ID NO: 1) against which an antibody was raised.

SEQ ID NO: 6 is the amino acid sequence of a mid-domain fragment of Aβ₁₋₄₂ and Aβ₁₋₄₀, namely, residues 20-34 of Aβ₁₋₄₂ (SEQ ID NO: 1) and Aβ₁₋₄₀ (SEQ ID NO: 2).

SEQ ID NO: 7 is the amino acid sequence of an epitope of Aβ₁₋₄₂ (SEQ ID NO: 1) and Aβ₁₋₄₀ (SEQ ID NO: 2) against which an antibody was raised.

SEQ ID NO: 8 is the amino acid sequence of an epitope of Aβ₁₋₄₂ (SEQ ID NO: 1) and Aβ₁₋₄₀ (SEQ ID NO: 2) against which an antibody was raised.

SEQ ID NO: 9 is the amino acid sequence of an epitope of Aβ₁₋₄₂ (SEQ ID NO: 1) and Aβ₁₋₄₀ (SEQ ID NO: 2) for detecting a mid-domain fragment of Aβ₁₋₄₂ (SEQ ID NO: 1) and Aβ₁₋₄₀ (SEQ ID NO: 2).

SEQ ID NO: 10 is the amino acid sequence of α-synuclein.

SEQ ID NO: 11 is the amino acid sequence of a mid-domain fragment of α-synuclein, namely residues 39-53 of α-synuclein.

SEQ ID NO: 12 is the amino acid sequence of a mid-domain fragment of α-synuclein, namely residues 53-72 of α-synuclein.

SEQ ID NO: 13 is the amino acid sequence of a mid-domain fragment of α-synuclein, namely residues 76-90 of α-synuclein.

SEQ ID NO: 14 is the amino acid sequence of Tau.

SEQ ID NO: 15 is the amino acid sequence of a mid-domain fragment of Tau, namely residues 306-322 of Tau.

SEQ ID NO: 16 is the amino acid sequence of a mid-domain fragment of Tau, namely residues 281-322 of Tau.

SEQ ID NO: 17 is the amino acid sequence of TDP-43.

SEQ ID NO: 18 is the amino acid sequence of a mid-domain fragment of TDP-43, namely residues 311-334 of TDP-43.

SEQ ID NO: 19 is the amino acid sequence of a mid-domain fragment of TDP-43, namely residues 246-255 of TDP-43.

SEQ ID NO: 20 is the amino acid sequence of myelin basic protein (MBP).

SEQ ID NO: 21 is the amino acid sequence of a mid-domain fragment of MBP, namely residues 30-70.

### DETAILED DESCRIPTION

The present invention arises because it has now, surprisingly, been shown that fragments of Aβ₁₋₄₂ (SEQ ID NO: 1), corresponding to so-called “mid-domain fragments”, such as residues 20-34 of Aβ₁₋₄₂ (SEQ ID NO: 5), can be used as a measure of MPS function or malfunction, Aβ₁₋₄₂ catabolism and clearance, and to assess the effect of different agents at different points of the MPS. In particular, it has now been shown that mid-domain fragments of Aβ₁₋₄₂ are resistant to catalysis and degradation (FIG. 2). As these mid-domain fragments retain toxicity and coincide with known familial mutations causing Alzheimer's disease (AD; FIG. 1), it is useful to measure and monitor catalysis and clearance of these fragments in order to monitor MPS function or malfunction, and to assess the effect of different agents on MPS As catalysis. It has also been found that mid-domain fragments of other proteins associated with neurodegeneration and MPS malfunction, wherein the mid-domain fragments are associated with aggregation and/or toxicity, are resistant to catalysis and clearance. Thus, it is expected that these mid-domain fragments can also be used as a measure of MPS function or malfunction, protein catabolism and clearance, and to assess the effect of different agents at different points of the MPS.

In addition, the relationship between inflammatory activation associated with AD and the function of the MPS can be assessed, as well as the ability of different agents to modulate the MPS on a background of inflammatory activation. In particular, it is now possible to assess how different agents modify both MPS inflammatory activation and Aβ₁₋₄₂ catabolism.

It has also, surprisingly, been found that live MPS cells obtained from a human subject can be used in the assessments of MPS function and inflammatory activation, and that this is beneficial for the development of personalised medicine for treating AD. In particular, MPS cells obtained from a human subject retain the same MPS malfunction as was present in vivo, due to these being caused by genetic and epigenetic changes. Thus, live MPS cells from a human subject can be used as a model of the MPS of that human subject, and the efficacy of different agents for treating that human subject can be assessed, to allow for selection of the most effective treatment for that subject.

As discussed above, it is also considered that similar assessments of the MPS can be carried out in relation to disease marker products, and fragments thereof, associated with neurodegenerative diseases and diseases with inflammatory activation, other than AD, which also show MPS malfunction. In particular, fragments of such other disease marker products, produced by catabolism by the MPS, are likely to retain toxicity and give disease impact, similar to the Aβ₁₋₄₂ mid-domain fragments.

### Methods

In general terms, the method is an ex vivo method of determining the effect of an agent on catalysis by a Monocyte Phagocyte System (MPS) cell of a disease marker product and/or at least one fragment thereof associated with neurodegeneration and/or inflammatory activation. The method comprises the steps of:
i) maintaining a sample of MPS cells under conditions in which the MPS cells remain alive,
ii) exposing the sample of MPS cells to an agent and a disease marker product, to permit phagocytosis of the disease marker product by the MPS cells,
iii) detecting the intracellular amount of the disease marker product and/or at least one fragment thereof in the sample of MPS cells, and
iv) comparing the intracellular amount of the disease marker product and/or the at least one fragment thereof to an intracellular amount of the same disease marker product and/or at least one fragment thereof measured in control MPS cells in the absence of the agent.

The effect of the agent on catalysis by MPS cells of the disease marker product and/or at least one fragment thereof is determined by the result of the comparison of step iv).

In some embodiments, the sub-steps of step ii) (i.e. exposing the sample of MPS cells to an agent and exposing the sample of MPS cells to a disease marker product) are performed simultaneously. In other embodiments, the sub-step of exposing the sample of MPS cells to an agent is carried out before exposing the sample of MPS cells to a disease marker product. In further embodiments, the sub-step of exposing the sample of MPS cells to an agent is carried out after exposing the sample of MPS cells to a disease marker product.

### MPS Cells

The sample of MPS cells and the control MPS cells, independently, may have been obtained from a cell bank or from a human subject. As shown in Example 5, the method of the present invention can be used to reliably detect a fragment of a disease marker product in MPS cells obtained from a human subject. When obtained from a human donor, the sample of MPS cells and the control MPS cells may, independently, have been isolated from a blood sample or a CSF sample. Preferably, the sample of MPS cells is obtained from a blood sample. Preferably, the sample of MPS cells and the control MPS cells have been obtained from the same source, for example, both have been obtained from a blood sample or a CSF sample from the same human subject. Thus, in some embodiments, both the sample of MPS cells and the control MPS cells have been obtained from a blood sample from a human subject. In particular, blood samples are easier and less invasive to collect than CSF samples.

Methods of isolating MPS cells from blood and CSF samples are well known in the art, for example, by flow cytometry, magnetic extraction, density gradient centrifugation, bead isolation, “RosetteSep”, adherence to plastic or the use of antibodies.

When the sample of MPS cells is obtained from a human subject, the human subject may be healthy or may have, or be suspected of having, a neurodegenerative disease and/or inflammatory activation. In some embodiments, the human subject may be suspected of having, or have been diagnosed as having, AD, Parkinson's disease (PD), Multiple Sclerosis, Front Temporal Dementia or Amyotrophic Lateral Sclerosis.

### Cell Types

The sample of MPS cells and control MPS cells may, independently, be brain or peripheral MPS cells. In some embodiments, the sample of MPS cells and the control MPS cells are, independently, macrophages, monocytes or microglial cells. Preferably, the sample of MPS cells and the control MPS cells are, independently, monocytes. The sample of MPS cells and the control MPS cells may be the same or different types, but, in preferred embodiments, both the sample of MPS cells and the control MPS cells are monocytes.

In some embodiments, the method of the present invention includes a step of differentiating the sample of MPS cells and/or the control MPS cells. In some embodiments, the step of differentiating the sample of MPS cells and/or control MPS cells takes place before exposing the cells to the disease marker product and/or at least one fragment thereof, and an agent, as appropriate. In embodiments where the MPS cells have been obtained from a human subject, the step of differentiating the MPS cells takes place after isolating the MPS cells from the sample obtained from the human subject, and before exposing the cells to the disease marker product and/or at least one fragment thereof, and an agent, as appropriate.

In some embodiments, human induced pluripotent stem cells (hiPSCs), obtained from a cell bank or from a human subject, are differentiated into MPS cells, such as microglia, monocytes or macrophages. hiPSCs obtained from a human subject are, preferably, derived from fibroblast cells obtained from the human subject. For example, hiPSCs may be differentiated into microglia. Alternatively, hiSPCs may be differentiated into monocytes and, optionally, further differentiated into macrophages. Methods of producing hiPSCs and differentiating hiPSCs into MPS cells are known, and any may be used in the method of the present invention.

In some embodiments, the sample of MPS cells and the control MPS cells are, independently, monocytes, and the method of the invention comprises a step of differentiating the monocytes into macrophages. The monocytes may have been derived from hiPSCs, as discussed above, or may have been obtained directly from a cell bank or a human subject. Any method known in the field for differentiating monocytes to macrophages may be used.

Maintaining the MPS Cells Under Conditions in which they Remain Alive

The sample of MPS cells and the control MPS cells are seeded in an appropriate medium for keeping the MPS cells alive. Such appropriate media are known in the art and may be, for example, RPMI 1640 or Iscove's Modified Dulbecco's Medium (IMBM). It is not necessary for the cells to grow, but it is required that the cells remain alive, so that the function of MPS, and the effect of exposure of the cells to any agents, can be effectively assessed. In addition, when the sample of MPS cells and/or the control MPS cells have been obtained from a human subject, any genetic or epigenetic changes affecting the MPS cells of the human subject in vivo are maintained in the sample of MPS cells and/or the control MPS cells in vitro, such that the sample of MPS cells and/or the control MPS cells provide a model of the MPS of the human subject.

When the method comprises a step of differentiating the sample of MPS cells and/or the control MPS cells, this differentiation step may take place before the step of exposing the respective MPS cells to the disease marker product and/or at least one fragment thereof, and. as appropriate, an agent. In other words the step of differentiating the MPS cells may take place before step ii) of the method. In some embodiments, the step of differentiating the cells occurs during the step of maintaining the sample of MPS cells and/or the control MPS cells under conditions in which they remain alive (step i) of the method). Thus, in some embodiments, step i) of the method comprises differentiating a sample of MPS cells, which are monocytes, into macrophages and maintaining the sample of MPS cells under conditions in which the MPS cells remain alive. Accordingly, the step of maintaining the MPS cells under conditions in which they remain alive applies to both the undifferentiated cells (i.e. monocytes) and the differentiated cells (e.g. macrophages).

In some embodiments, the step of differentiating the sample of MPS cells and/or the control MPS cells, which are monocytes, to macrophages comprises exposing the sample of MPS cells and/or the control MPS cells to 12-O-tetradecanoylphorbol 13-acetate (TPA) or macrophage colony-stimulating factor (MCSF) before exposure to the disease marker product and/or at least one fragment thereof, and, as appropriate, the agent. Preferably, when the monocytes are hiPSC-derived monocytes, the monocytes are exposed to MCSF in order to differentiate them to macrophages. In some embodiments, the cells are exposed to TPA 0-14 hours before they are exposed to the disease marker product and/or at least one fragment thereof, and, as appropriate, the agent. When the cells are exposed to TPA 0 hours before they are exposed to the disease marker product and/or at least one fragment thereof, and, as appropriate, the agent, this means that TPA is added to the cells immediately before the step of exposing the cells to the disease marker product and/or at least one fragment thereof, and, as appropriate, the agent.

In some embodiments, the method of the present invention comprises a step of culturing the sample of MPS cells and/or the control MPS cells, in order to allow the cells to grow.

The sample of MPS cells and/or the control MPS cells may be maintained alive, or cultured, for up to 48 hours, up to 36 hours, up to 24 hours, up to 12 hours, up to 11 hours, up to 10 hours, up to 9 hours, up to 8 hours, up to 7 hours, up to 6 hours, up to 5 hours, up to 4 hours, up to 3 hours, up to 2 hours or up to 1 hour. In some embodiments, the sample of MPS cells and/or the control MPS cells are maintained alive, or cultured, for a minimum of 30 minutes or a minimum of 1 hour. In some embodiments, the sample of MPS cells and/or the control MPS cells are maintained alive, or cultured, for 1, 2, 3, 4, 5, 6, 7, 8, 9, or 10 hours, preferably 6 hours. In some embodiments, the sample of MPS cells are exposed to the agent and the disease marker product and/or at least one fragment thereof, and control MPS cells are exposed to the disease marker product and/or at least one fragment thereof, immediately after the cells have been obtained.

The sample of MPS cells and/or the control MPS cells may be frozen, and subsequently thawed, prior to being maintained in conditions under which the cells remain alive.

### Exposure to the Agent and to the Disease Marker Product

The sample of MPS cells is exposed to an agent and to a disease marker product and/or at least one fragment thereof. In some embodiments, the sample of MPS cells is exposed to the agent before it is exposed to the disease marker product and/or at least one fragment thereof.

The control MPS cells are exposed to the disease marker product and/or at least one fragment thereof, but not to the agent.

Exposure of the sample of MPS cells and/or the control MPS cells preferably occurs under conditions in which the cells are maintained alive.

In some embodiments, the method comprises a step of culturing the sample of MPS cells and/or the control MPS cells during exposure to the agent and/or the disease marker product and/or the at least one fragment thereof, as appropriate.

The sample of MPS cells and/or the control MPS cells are exposed to the disease marker product and/or at least one fragment thereof for 1-4 hours, preferably for 2 hours.

The sample of MPS cells is exposed to the agent for 1-16 hours, preferably 4 hours.

The sample of MPS cells and/or the control MPS cells may be exposed to the disease marker product and/or at least one fragment thereof at a concentration of 10-1000 ng/ml, 10-750 ng/ml, 50-750 ng/ml, 50-600 ng/ml, 50-500 ng/ml, 100-500 ng/ml, 100-250 ng/ml, 100-200 ng/ml or 100-150 ng/ml. In some embodiments, the disease marker product and/or at least one fragment thereof is at a concentration of 100 ng/ml or 500 ng/ml. In some embodiments, the concentration of the disease marker product and/or at least one fragment thereof is 100 ng/ml. When more than one of a disease marker product and/or at least one fragment thereof is used (e.g. a disease marker product and one fragment thereof, or two fragments of a disease marker product), each may be at a concentration as set out above.

The sample of MPS cells may be exposed to the agent at a concentration of 50 nM-200 μM, 50 nM-150 μM, 50 nM-100 μM, 100 nM-100 μM, 200 nM-100 μM, 100 nM-1 μM or 200 nm-1 μM. In some embodiments, the agent is at a concentration of 100 nM, 200 nM, 1 μM, 10 μM or 100 μM.

The sample of MPS cells may be exposed to an agent at a concentration of 50-200 ng/ml, 50-150 ng/ml, 75-150 ng/ml or 75-100 ng/ml. In some embodiments, the agent is at a concentration of 50 ng/ml, 100 ng/ml or 150 ng/ml.

Detection of the Disease Marker Product and/or at Least One Fragment Thereof

The step of detecting the amount of intracellular disease marker product and/or at least one fragment thereof in the samples of MPS cells and/or the control MPS cells can be carried out using any techniques known in the art. For example, the step of detecting the disease marker product and/or at least one fragment thereof may be using an immunoassay, Mass Spectrometry techniques, semi-quantitative techniques such as Western Blot, microscopy, flow cytometry, or ELISA techniques. While the sample of MPS cells and/or the control MPS cells are maintained under conditions in which the cells are kept alive during exposure to the agent and or the disease marker product and/or at least one fragment thereof, the step of detecting the amount of intracellular disease marker product and/or at least one fragment thereof typically requires lysis of the cells.

The step of detecting the disease marker product and/or at least one fragment thereof can detect any part of the disease marker product and/or fragment thereof. In some embodiments, the detection comprises detecting one end of the disease marker product and/or a fragment thereof, for example, the N-terminal or the C-terminal of a disease marker product and/or a fragment thereof which is a protein or peptide.

In some embodiments, the method may comprise detecting at least one fragment of the disease marker product. In some embodiments, the method comprises detecting two or more fragments of the disease marker product. Preferably, the method comprises detecting two, and more preferably three, fragments of the disease marker product.

The step of detecting the disease marker product and/or at least one fragment thereof may use any suitable detection technique known in the art. In some embodiments, the step of detecting the intracellular disease marker product and/or at least one fragment thereof uses antibody detection.

The method of the invention may comprise steps of washing the samples of MPS cells and/or the control MPS cells, and producing a cell lysate, before the step of detecting the intracellular amount of the disease marker product and/or at least one fragment thereof. The method may additionally or alternatively, comprise a step of freezing the lysate, for example, at −80° C., before the step of detecting the intracellular amount of the disease marker product and/or at least one fragment thereof. If the lysate is frozen before the detecting step, then the lysates are defrosted before the step of detecting the intracellular amount of the disease marker product and/or at least one fragment thereof is carried out.

### Agent

The agent to which the sample of MPS cells is exposed may be a known or suspected MPS modulator (e.g. a stimulator or an inhibitor) or inflammatory activator. A MPS modulator modulates the MPS autophagic and/or endolysosomal systems. In some embodiments, the agent to which the sample of MPS cells is exposed is a pharmaceutical composition comprising the agent or a pharmaceutically-acceptable salt thereof.

In some embodiments, the MPS modulator is Rapamycin, a Rapamycin analogue (so-called “Rapalogues”), a FFAR4 agonist, an α7 nAChR agonist or positive allosteric modulator (PAM), Bafilomycin, IDE inhibitor, Neprilysin inhibitor or BACE inhibitor, or a combination of two or more thereof.

In some embodiments, the MPS modulator is a MPS stimulator. In some embodiments, the MPS stimulator is Rapamycin, a Rapamycin analogue (so-called “Rapalogues”), a FFAR4 agonist or an α7 nAChR agonist or positive allosteric modulator (PAM), or a combination of two or more thereof. In some embodiments, the agent is a combination of a FFAR4 agonist and a α7 nAChR agonist or PAM. In some embodiments, the agent is Rapamycin, PUFA such as DHA, NS1738 or a combination of DHA and NS1738. In particular, Rapamycin is known to enhance macroautophagy (Spilman et al, 2010; Malik et al., 2019), and FIG. 4 shows that Rapamycin increases clearance of the Aβ fragment Aβₓ₋₃₄. In addition, DHA has been shown to increase Aβ₁₋₄₂ phagocytosis and degradation (WO 2020/212627). FIG. 6 shows that exposure of MPS cells to DHA alone reduces the amount of the Aβ fragment Aβₓ₋₃₄, and that DHA administered in combination with the α7 nAChR PAM NS1738 significantly reduces the amount of all three of the Aβ fragments Aβ₂₀₋ₓ, Aβₓ₋₃₄ and Aβₓ₋₄₀. In addition, treatment with the α7 nAChR PAM NS1738 alone reduces the amount of Aβ fragment Aβₓ₋₃₄. Thus, Rapamycin, DHA and/or NS1738 can be used as agents for assessing MPS function, and particularly for assessing the MPS function of a specific human subject and the modulation of the human subject's MPS function to different treatments. Furthermore, FIG. 8 shows that DHA and NS1738 continue to have an effect on the levels of the three Aβ fragments, Aβ₂₀₋ₓ, Aβₓ₋₃₄ and Aβₓ₋₄₀, in the presence of inflammatory activation induced by LPS, such that the methods of the present invention can be used to assess the effect of agents on MPS function on a background of inflammatory activation.

In some embodiments, the MPS modulator is a MPS inhibitor. In some embodiments, the MPS inhibitor is Bafilomycin, IDE inhibitor, Neprilysin inhibitor or BACE inhibitor, or a combination of two or more thereof. In some embodiments, the MPS inhibitor is a combination of IDE inhibitor, Neprilysin inhibitor and BACE inhibitor. In particular, FIGS. 4 and 5 show that these MPS inhibitors increase the amount of the Aβ fragments Aβ₂₀₋ₓ and Aβ₁₋₄₀, and that different inhibitors affect the levels of different Aβ fragments. Thus, MPS inhibitors can be used to assess MPS function at different points thereof.

The agent may alternatively be an inflammatory activator. In some embodiments, the inflammatory activator is lipopolysaccharide (LPS), TPA or a Damage-Associated Molecule Pattern (DAMP), such as Biglycan, hyaluronan, Heparan sulphate, Aβ₁₋₄₂, Aβ₁₋₄₀, or a fragment of Aβ₁₋₄₂ or Aβ₁₋₄₀. In particular, it has been shown that Aβ modulates proinflammatory functions and that MCP-1/JE, a proinflammatory cytokine, is released in response to Aβ (Meda et al., 1996). In some embodiments the agent is Aβ₁₋₄₂, Aβ₁₋₄₀ or a fragment of Aβ₁₋₄₂ or Aβ₁₋₄₀, and preferably is Aβ₁₋₄₂ or Aβ₁₋₄₀. In these embodiments, the disease marker product may be any of Aβ₁₋₄₂, Aβ₁₋₄₀ or any fragment of Aβ₁₋₄₂ or Aβ₁₋₄₀ described below. Preferably, the inflammatory activator is LPS.

In some embodiments, the agent is a MPS stimulator or a MPS inhibitor, and the step of exposing the sample MPS cells to an agent additionally comprises exposing the sample MPS cells to an inflammatory activator. In some embodiments, the sample of MPS cells is exposed to a MPS stimulator or a MPS inhibitor, and to an inflammatory activator, and the control MPS cells are exposed to the same MPS stimulator or MPS inhibitor, but not to an inflammatory activator. In some embodiments, the sample of MPS cells is exposed to a MPS stimulator or a MPS inhibitor, and to an inflammatory activator, and the control MPS cells are exposed to the same inflammatory activator, but not to a MPS stimulator or a MPS inhibitor. In some embodiments, the sample of MPS cells is exposed to a MPS stimulator or a MPS inhibitor, and to an inflammatory activator, and the control MPS cells are not exposed to any of MPS stimulator, a MPS inhibitor and an inflammatory activator.

As shown in FIG. 8, therapeutic interventions can be assessed for their effect on the MPS in the context of inflammatory activation. This provides the advantage of being able to assess the MPS function, and the effect of agents, such as candidate therapeutic interventions, thereon, on an inflammatory background. This provides a more accurate model of in vivo MPS function. In addition, the method of the present invention means that conditions associated with inflammatory activation and MPS malfunction, other than neurodegenerative conditions, can be studied by detecting fragments of an associated disease marker product which result from clearance by the MPS. The effect of candidate interventions on such inflammatory conditions on MPS function can also be assessed.

In some embodiments, the method comprises exposing a sample of MPS cells from the same source to a panel of agents, and comparing the effect of each agent on MPS function. This may involve separating the sample of MPS cells into multiple sub-samples. Thus, in some embodiments, the method comprises maintaining more than one sample of MPS cells under conditions in which the MPS cells remain alive, exposing each sample of MPS cells to the same disease marker product and/or at least one fragment thereof, detecting the intracellular amount of the disease marker product and/or at least one fragment thereof in each sample of MPS cells, exposing each sample of MPS cells to a different agent, and comparing the intracellular amount of the disease marker product and/or the at least one fragment thereof in each sample of MPS cells to one another as well as to control MPS cells in the absence of any agent. Thus, the method of the invention can be for identifying which agent, amongst a panel of agents, has the greatest effect on MPS function (i.e. catalysis of the disease marker product and/or at least one fragment thereof) or modifies the MPS function in a desirable way.

### Comparison of the Sample of MPS Cells and the Control MPS Cells

The amount of intracellular disease marker product and/or at least one fragment thereof detected in the sample of MPS cells is compared to the amount of intracellular disease marker product and/or at least one fragment thereof detected in the control MPS cells. The difference between the two amounts indicates the effect of the agent administered to the sample of MPS cells on the MPS and catalysis of the disease marker product and/or at least one fragment thereof, thereby providing an indication of MPS functionality. The type of fragment detected can also provide an indication of MPS functionality, and, specifically, the part of the MPS which is affected by the neurodegeneration and/or inflammatory activation. Thus, the amount of disease marker product and/or at least one fragment thereof, as well as the type of fragment of the disease marker product, detected indicates the effect of the agent to which the sample of MPS cells has been exposed. In particular, the difference in amount of the disease marker product and/or at least one fragment thereof is indicative of the specific endolysosomal enzyme affected by the agent to which the sample of MPS cells has been exposed. In addition, when the method comprises a step of exposing the sample of MPS cells and/or the control MPS cells to an inflammatory activator, the difference in the amounts of intracellular disease marker product and/or at least one fragment thereof can also indicate how the agent interacts with inflammatory activation to affects the MPS.

### Disease Marker Products and Fragments Thereof

The disease marker product and/or at least one fragment thereof is associated with neurodegeneration and/or inflammatory activation. In some embodiments, the disease marker product, and associated neurodegeneration and/or inflammatory activation, is associated with MPS malfunction and abnormal clearance of the disease marker product and/or at least one fragment thereof. Preferably, the neurodegeneration is associated with inflammatory activation. In some embodiments, the disease marker product and/or at least one fragment thereof is associated with an inflammatory condition or disease, an autoimmune disease or an infectious disease, such as an RNA virus infection. In some embodiments, the neurodegeneration and/or inflammatory activation, and the relevant associated disease marker product, is one of the following:
AD: Aβ₁₋₄₂, Aβ₁₋₄₀, Tau protein or α-synuclein,
Amyloid angiopathy: Aβ₁₋₄₂, Aβ₁₋₄₀, basal lamina proteins, lipids and lipoproteins,
PD: ubiquitin, Tau protein or α-synuclein,
Multiple Sclerosis: myelin basic protein,
Fronto Temporal Dementia: Tau protein or TDP-43,
Amyotrophic Lateral Sclerosis: Tau protein or TDP-43,
Lewy Body Dementia: α-synuclein,
Vascular degenerative diseases with inflammatory components, including vascular dementia (amyloid angiopathies, atherosclerosis and arteriolosclerosis): basal lamina proteins, lipids and lipoproteins,
Multiple System Atrophy: α-synuclein,
Progressive Supranuclear Palsy: Tau protein,
RNA virus infection: capsid proteins.

In some embodiments, a disease marker product and at least one fragment thereof is detected. In some embodiments, one or more fragments of the disease marker product is detected, for example, two fragments or three fragments. When more than one fragment is detected, two or more of the fragments may comprise overlapping stretches of amino acids (i.e. two or more of the fragments comprise a stretch of amino acids which are common to the fragments). Alternatively, when more than one fragment is detected, two or more fragments may comprise stretches of amino acids which do not overlap between the two or more fragments (i.e. the two or more fragments do not contain common stretches of amino acids). This is advantageous for assessing the effect of an agent on different points of MPS function and catabolism.

In some embodiments, two or more different disease marker products are detected. In some embodiments, a disease marker product and at least one fragment of a different disease marker product are detected. In some embodiments, two or more fragments of different disease marker products are detected. Each of the disease marker products, and/or at least one fragment thereof, may be selected from the list above. In these embodiments, the sample of MPS cells and/or the control MPS cells are exposed to two or more different disease marker products and/or fragments thereof either in a single, multiplexed test or in separate tests on sub-samples, and the step of comparing the intracellular amount (i.e. step iv) comprises comparing the intracellular amount of each disease marker product and/or at least one fragment thereof in the sample of MPS cells to the intracellular amount of the same or corresponding disease marker product and/or at least one fragment thereof in control MPS cells.

In some embodiments, separate samples of MPS cells are used for each of the disease marker product and/or fragment thereof, as appropriate. In some embodiments, the detection of the disease marker product(s) and/or each fragment thereof, as appropriate, is multiplexed, to permit simultaneous detection.

Aβ

In some embodiments, the disease marker product is Aβ₁₋₄₂ (SEQ ID NO: 1) or Aβ₁₋₄₀ (SEQ ID NO: 2), preferably Aβ₁₋₄₀ (SEQ ID NO: 2). In particular, the Examples show that several different fragments of Aβ₁₋₄₀ (SEQ ID NO: 2) can be detected, and the effect of different agents on clearance thereof can be assessed. In these embodiments, therefore, where the sample of MPS cells is obtained from a human subject suspect of having, or diagnosed as having, a neurodegenerative disease, the neurodegenerative disease is AD. In particular, and as discussed above, shorter fragments of Aβ₁₋₄₂ and Aβ₁₋₄₀ retain toxicity, and, therefore, represent a crossroad in catalysis of Aβ, which can be measured. Aβ₁₋₄₂ and Aβ₁₋₄₀ are known to be catabolised to mid-domain fragments, by cleavage for example between residues 18 and 19, residues 19 and 20, residues 20 and 21, residues 33 and 34 and/or residues 34 and 35 (Rogeberg et al., 2015; Henjum et al., 2020), such that it is possible to detect catalysis products by detection of the N- and/or C-terminals of the mid-domain fragments. As these mid-domain fragments are less susceptible to catalysis by the MPS, it is expected that these mid-domain fragments are retained intracellularly in MPS cells and are therefore detectable. It is, therefore, possible to assess the function and efficacy of the MPS in clearance of Aβ, as well as the effects of different agents thereon and the interaction with inflammation, as discussed above.

Thus, in some embodiments, the method comprises detecting at least one fragment of Aβ₁₋₄₂ (SEQ ID NO: 1) or Aβ₁₋₄₀ (SEQ ID NO: 2). In some embodiments, the method comprises detecting more than one fragment of Aβ₁₋₄₂ (SEQ ID NO: 1) or Aβ₁₋₄₀ (SEQ ID NO: 2). In some embodiments, the method comprises detecting two, and preferably three, fragments of Aβ₁₋₄₂ (SEQ ID NO: 1) or Aβ₁₋₄₀ (SEQ ID NO: 2).

The method can comprise detecting at least one fragment of Aβ₁₋₄₂ (SEQ ID NO: 1) or Aβ₁₋₄₀ (SEQ ID NO: 2) which is Aβ₁₋₄₂ (SEQ ID NO: 1) or Aβ₁₋₄₀ (SEQ ID NO: 2) which has been cleaved between amino acid residues 19 and 20 thereof and/or between amino acid residues 34 and 35 thereof.

In some embodiments, the at least one fragment is Aβ₁₋₄₂ (SEQ ID NO: 1) or Aβ₁₋₄₀ (SEQ ID NO: 2) which has been cleaved between amino acid residues 19 and 20 thereof. These fragments are referred to herein as “Aβ₂₀₋ₓ”. In these embodiments, the at least one fragment may be detected by detecting a region of the fragment which comprises at least 4 amino acids including residue 21 of Aβ₁₋₄₂ (SEQ ID NO: 1) or Aβ₁₋₄₀ (SEQ ID NO: 2). The at least one fragment may be detected by detecting a region of the fragment comprising residues 21-24, preferably by detecting residues 21-29, of Aβ₁₋₄₂ (SEQ ID NO: 1) or Aβ₁₋₄₀ (SEQ ID NO: 2). In some embodiments, the at least one fragment may be detected by detecting the N-terminal of SEQ ID NO: 6. In some embodiments, the at least one fragment is detected by detecting any one of the aforementioned regions comprising residue 21 or detecting the N-terminal of SEQ ID NO: 6 and, in addition, detecting a region of the fragment which comprises at least 4 amino acid residues including residue 34 or residue 40 of Aβ₁₋₄₂ (SEQ ID NO: 1) or Aβ₁₋₄₀ (SEQ ID NO: 2), preferably a region of the fragment comprising residues 34-40 of Aβ₁₋₄₂ (SEQ ID NO: 1) or Aβ₁₋₄₀ (SEQ ID NO: 2), and more preferably detecting SEQ ID NO: 9. In some embodiments, the at least one fragment is detected by detecting a region of the fragment comprising residues 21-24 of AB₁₋₄₂ (SEQ ID NO: 1) or Aβ₁₋₄₀ (SEQ ID NO: 2) and detecting a region of the fragment comprising residues 34-40 of Aβ₁₋₄₂ (SEQ ID NO: 1) or Aβ₁₋₄₀ (SEQ ID NO: 2). In some embodiments, the at least one fragment is detected by detecting the N-terminal of SEQ ID NO: 3 and a region of the fragment comprising residues 34-40 of Aβ₁₋₄₂ (SEQ ID NO: 1) or Aβ₁₋₄₀ (SEQ ID NO: 2), preferably by detecting the N-terminal of SEQ ID NO: 6 and detecting SEQ ID NO: 9. In some embodiments, the at least one fragment is detected using an antibody which is specific to the N-terminal of SEQ ID NO: 6, preferably using an antibody is specific to SEQ ID NO: 3. In some embodiments, the at least one fragment is detected using an antibody specific to the region of the fragment comprising residues 34-40 of Aβ₁₋₄₂ (SEQ ID NO: 1) or Aβ₁₋₄₀ (SEQ ID NO: 2), preferably using an antibody specific to SEQ ID NO: 9. In some embodiments, the at least one fragment is detected using an antibody specific to the N-terminal of SEQ ID NO: 6 and an antibody specific to SEQ ID NO: 9.

In some embodiments, the at least one fragment is Aβ₁₋₄₂ (SEQ ID NO: 1) or Aβ₁₋₄₀ (SEQ ID NO: 2) which has been cleaved between amino acid residues 34 and 35 thereof. These fragments are referred to herein as “Aβₓ₋₃₄”. In these embodiments, the at least one fragment may be detected by detecting a region of the fragment which comprises at least 4 amino acids including residue 34 of Aβ₁₋₄₂ (SEQ ID NO: 1) or Aβ₁₋₄₀ (SEQ ID NO: 2). The at least one fragment may be detected by detecting a region of the fragment comprising residues 31-34 of Aβ₁₋₄₂ (SEQ ID NO: 1) or Aβ₁₋₄₀ (SEQ ID NO: 2), preferably by detecting the C-terminal of SEQ ID NO: 6. In some embodiments, the at least one fragment is detected by detecting any one of the aforementioned regions comprising residue 34 or the C-terminal of SEQ ID NO: 6 and in addition detecting a region of the fragment comprising at least 4 amino acids including residue 21 of Aβ₁₋₄₂ (SEQ ID NO: 1) or Aβ₁₋₄₀ (SEQ ID NO: 2), preferably a region of the fragment comprising residues 21-24 of Aβ₁₋₄₂ (SEQ ID NO: 1) or Aβ₁₋₄₀ (SEQ ID NO: 2), and more preferably a region of the fragment comprising residues 21-29 of Aβ₁₋₄₂ (SEQ ID NO: 1) or Aβ₁₋₄₀ (SEQ ID NO: 2). In some embodiments, the at least one fragment is detected by detecting the C-terminal of SEQ ID NO: 6 and detecting a region of the fragment comprising residues 21-24 of Aβ₁₋₄₂ (SEQ ID NO: 1) or Aβ₁₋₄₀ (SEQ ID NO: 2), more preferably by detecting the C-terminal of SEQ ID NO: 6 and detecting a region of the fragment comprising residues 21-29 of Aβ₁₋₄₂ (SEQ ID NO: 1) or Aβ₁₋₄₀ (SEQ ID NO: 2). In some embodiments, the at least one fragment is detected by detecting the C-terminal of SEQ ID NO: 6 and detecting SEQ ID NO: 7. In some embodiments, the at least one fragment is detected using an antibody which is specific to the C-terminal of SEQ ID NO: 6, preferably using an antibody which is specific for SEQ ID NO: 4. In some embodiments, the at least one fragment is detected using an antibody which is specific to the region of the fragment comprising at least 4 amino acids comprising residues 21-24 of Aβ₁₋₄₂ (SEQ ID NO: 1) or Aβ₁₋₄₀ (SEQ ID NO: 2), preferably an antibody specific to SEQ ID NO: 7. In some embodiments, the at least one fragment is detected using an antibody specific to the C-terminal of SEQ ID NO: 6 and an antibody specific to SEQ ID NO: 7.

In some embodiments, the at least one fragment is Aβ₁₋₄₂ (SEQ ID NO: 1) or Aβ₁₋₄₀ (SEQ ID NO: 2) which has been cleaved between amino acid residues 19 and 20, and between amino acid residues 34 and 35, such that the at least one fragment has the amino acid sequence shown in SEQ ID NO: 6. In these embodiments, the at least one fragment may be detected by detecting the N- or the C-terminal of SEQ ID NO: 6. In some embodiments, the at least one fragment may be detected by detecting the N- and the C-terminal of SEQ ID NO: 6. In some embodiments, the at least one fragment is detected using an antibody which is specific for the N- or the C-terminal of SEQ ID NO: 6, preferably an antibody which is specific for SEQ ID NO: 3 or SEQ ID NO: 4. In some embodiments, the at least one fragment may be detected using an antibody specific for the N-terminal of SEQ ID NO: 6 and an antibody which binds a fragment of Aβ₁₋₄₂ (SEQ ID NO: 1) or Aβ₁₋₄₀ (SEQ ID NO: 2) comprising residue 34 of Aβ₁₋₄₂ (SEQ ID NO: 1) or Aβ₁₋₄₀ (SEQ ID NO: 2) at its C-terminal. In some embodiments, the at least one fragment is detected using an antibody specific for the N-terminal of SEQ ID NO: 6 and using an antibody specific for the C-terminal of SEQ ID NO: 6. In some embodiments, the at least one fragment is detected using an antibody which is specific for SEQ ID NO: 3 and an antibody specific for SEQ ID NO: 4.

In some embodiments, the method comprises the step of detecting Aβ₁₋₄₂ (SEQ ID NO: 1) or Aβ₁₋₄₀ (SEQ ID NO: 2), or at least one fragment of fragment thereof, by detecting the N-terminus of SEQ ID NO: 1 or SEQ ID NO: 2. In some embodiments, the method comprises the step of detecting Aβ₁₋₄₂ (SEQ ID NO: 1) or Aβ₁₋₄₀ (SEQ ID NO: 2), or at least one fragment of fragment thereof, by detecting the C-terminus of SEQ ID NO: 1 or SEQ ID NO: 2. These peptides and fragments thereof are referred to herein as “Aβₓ₋₄₀”.

In these embodiments, the detection may be by detecting a region of the fragment comprising at least 4 amino acids including residue 40 of Aβ₁₋₄₂ (SEQ ID NO: 1) or Aβ₁₋₄₀ (SEQ ID NO: 2). In some embodiments, the at least one fragment may be detected by detecting a region of at least 4 amino acids comprising residues 39-40 of Aβ₁₋₄₂ (SEQ ID NO: 1) or Aβ₁₋₄₀ (SEQ ID NO: 2), preferably by detecting the C-terminal of SEQ ID NO: 2. In some embodiments, the at least one fragment may be detected by detecting a region of the fragment comprising residues 39-42, 34-40 or 34-41 of Aβ₁₋₄₂ (SEQ ID NO: 1) or a region of the fragment comprising residues 34-40 of Aβ₁₋₄₀ (SEQ ID NO: 2). In some embodiments, the at least one fragment may be detected by detecting any one of the aforementioned regions comprising residue 40 or the C-terminal of SEQ ID NO: 2 and, in addition, detecting a region of the fragment comprising at least 4 amino acids including residue 21 of Aβ₁₋₄₂ (SEQ ID NO: 1) or Aβ₁₋₄₀ (SEQ ID NO: 2), preferably a region of the fragment comprising residues 21-24 of Aβ₁₋₄₂ (SEQ ID NO: 1) or Aβ₁₋₄₀ (SEQ ID NO: 2), and more preferably a region of the fragment comprising residues 21-29 of Aβ₁₋₄₂ (SEQ ID NO: 1) or Aβ₁₋₄₀ (SEQ ID NO: 2). In some embodiments, the at least one fragment is detected by detecting the C-terminal of SEQ ID NO: 2 and detecting a region of the fragment comprising residues 21-24 of Aβ₁₋₄₂ (SEQ ID NO: 1) or Aβ₁₋₄₀ (SEQ ID NO: 2), more preferably the C-terminal of SEQ ID NO: 2 and a region of the fragment comprising residues 21-29 of Aβ₁₋₄₂ (SEQ ID NO: 1) or Aβ₁₋₄₀ (SEQ ID NO: 2). In some embodiments, the at least one fragment may be detected by detecting a region of the fragment comprising residues 39-42, 34-40 or 34-41 of Aβ₁₋₄₂ (SEQ ID NO: 1) or a region of the fragment comprising residues 34-40 of Aβ₁₋₄₀ (SEQ ID NO: 2) and detecting a region of the fragment comprising residues 21-29 of Aβ₁₋₄₂ (SEQ ID NO: 1) or Aβ₁₋₄₀ (SEQ ID NO: 2). In some embodiments, the at least one fragment may be detected using an antibody which is specific for any one of the above-mentioned regions or for the C-terminus of SEQ ID NO: 1 or SEQ ID NO: 2. In some embodiments, the at least one fragment is detected using an antibody which is specific for SEQ ID NO: 5, an antibody which is specific for SEQ ID NO: 8 or an antibody which is specific for SEQ ID NO: 9. In some embodiments, the antibody specific SEQ ID NO: 9 is also specific for SEQ ID NO: 8 and/or SEQ ID NO: 5. In some embodiments, the antibody specific for SEQ ID NO: 8 is also specific for SEQ ID NO: 5, and vice versa. In some embodiments, the at least one fragment may be detected using an antibody which is specific for SEQ ID NO: 7 and an antibody specific for SEQ ID NO: 5, SEQ ID NO: 8 or SEQ ID NO: 9.

In some embodiments, the method comprises the step of detecting Aβ₂₀₋ₓ, Aβₓ₋₃₄, and Aβₓ₋₄₀. In other embodiments, the method comprises the step of detecting Aβ₂₀₋ₓ and Aβₓ₋₃₄; Aβ₂₀₋ₓ and Aβₓ₋₄₀; or Aβₓ₋₃₄ and Aβₓ₋₄₀. Each of these fragments can be detected using any of the methods described above. In some embodiments, each of these fragments is detected using antibodies specific for the N- or C-terminal of each, as described above.

In some embodiments, each of these fragments is detected using an antibody specific for SEQ ID NO: 3 or SEQ ID NO: 7, an antibody specific for SEQ ID NO: 4 and an antibody specific for SEQ ID NO: 5, SEQ ID NO: 8 or SEQ ID NO: 9. This is beneficial for assessing the efficacy of the MPS in clearing different fragments of Aβ, and assessing how different endolysosomal enzymes are affected by the agent.

In methods using antibodies to detect the disease marker product and/or at least one fragment thereof, a more generic antibody specific for Aβ₁₋₄₀ (SEQ ID NO: 2) or Aβ₁₋₄₂ (SEQ ID NO: 1) may be used as part of the detection method.

In some embodiments, the disease marker product is α-synuclein (SEQ ID NO: 10), Tau (SEQ ID NO: 14), TDP-43 (SEQ ID NO: 17) or MBP (SEQ ID NO: 19). In particular, it has been found that there is a region of each of α-synuclein, Tau and MBP which is expected to be less susceptible to cleavage by endolysosomal enzymes (FIGS. 11-14), and these regions are associated with regions previously believed to be important in aggregation and/or toxicity in neurodegenerative diseases and MPS malfunctions (discussed above). It is therefore now expected that these mid-domain fragments are retained intracellularly in MPS cells and can be detected, thereby making these fragments useful for assessing the function or malfunction of the MPS and the effect of different agents on the MPS and protein catabolism and clearance. In addition, it is known that several regions of TDP-43 are important for aggregation (Saini & Chauhan, 2011; Cao et al., 2019), and, in view of the association of degradation-resistant fragments of Aβ₁₋₄₂, Aβ₁₋₄₀, α-synuclein, Tau and MBP with fragments associated with aggregation and/or toxicity, it is now expected that these regions of TDP-43 are also resistant to degradation. It is therefore expected that these mid-domain fragments of TDP-43 can be detected in order to monitor and measure the function or malfunction of the MPS and the effect of different agents on the MPS and protein catabolism and clearance.

α-Synuclein

In some embodiments, the disease marker product is α-synuclein (SEQ ID NO: 10), and the method comprises detecting α-synuclein (SEQ ID NO: 10) or at least one fragment thereof. In some embodiments, the detection may be of α-synuclein (SEQ ID NO: 10) which has been cleaved between amino acid residues 38 and 39, and/or between amino acid residues 53 and 54. In some embodiments, the at least one fragment may be detected by detecting a region of the fragment comprising at least 4 amino acids comprising residue 39, preferably a region comprising residues 39-42, of α-synuclein (SEQ ID NO: 10). In some embodiments, the at least one fragment may be detected by detecting the N-terminal of SEQ ID NO: 11. In some embodiments, the at least one fragment may be detected by detecting a region of the fragment comprising at least 4 amino acids comprising residue 53 of α-synuclein (SEQ ID NO: 10), preferably a region of the fragment comprising residues 50-53 of α-synuclein (SEQ ID NO: 10). In some embodiments, the at least one fragment may be detected by detecting the C-terminal of SEQ ID NO: 11. In some embodiments, the at least one fragment may be detected by detecting a region of the fragment comprising at least 4 amino acids comprising residue 39, preferably comprising residues 39-42, of α-synuclein (SEQ ID NO: 10) and, in addition, detecting a region of the fragment comprising at least 4 amino acids comprising residue 53 of α-synuclein (SEQ ID NO: 10), preferably a region of the fragment comprising residues 50-53 of α-synuclein (SEQ ID NO: 10). In some embodiments, the at least one fragment may be detected by detecting the N-terminal of SEQ ID NO: 11 and detecting a region of the fragment comprising at least 4 amino acids comprising residue 53 of α-synuclein (SEQ ID NO: 10), preferably a region comprising residues 50-53, of α-synuclein (SEQ ID NO: 10). In some embodiments, the at least one fragment may be detected by detecting the C-terminal of SEQ ID NO: 11 and detecting a region of the fragment comprising at least 4 amino acids comprising residue 39 of α-synuclein (SEQ ID NO: 10), preferably a region of the fragment comprising residues 39-42 of α-synuclein (SEQ ID NO: 10). In some embodiments, the at least one fragment may be detected by detecting the N- and C-terminals of SEQ ID NO: 11. The at least one fragment may be detected using an antibody specific for any of the aforementioned regions or C- or N-terminals. In some embodiments, the at least one fragment may be detected using an antibody specific for a region of the fragment comprising at least 4 amino acids comprising residue 39, preferably comprising residues 39-42, of α-synuclein (SEQ ID NO: 10), or specific for the N-terminal of SEQ ID NO: 11, and an antibody specific for a region of the fragment comprising at least 4 amino acids comprising residue 53 of α-synuclein (SEQ ID NO: 10), preferably a region of the fragment comprising residues 50-53 of α-synuclein (SEQ ID NO: 10), or specific for the C-terminal of SEQ ID NO: 11. In some embodiments, the at least one fragment may be detected using an antibody specific for the N-terminal of SEQ ID NO: 11 and using an antibody specific for a region of the fragment comprising at least 4 amino acids comprising residue 53 of α-synuclein (SEQ ID NO: 10), preferably a region of the fragment comprising residues 50-53 of α-synuclein (SEQ ID NO: 10). In some embodiments, the at least one fragment may be detected using an antibody specific for the C-terminal of SEQ ID NO: 11 and an antibody specific for a region of the fragment comprising at least 4 amino acids comprising residue 39, preferably comprising residues 39-42, of α-synuclein (SEQ ID NO: 10). In some embodiments, the at least one fragment may be detected using an antibody specific for the N-terminal of SEQ ID NO: 11 and an antibody specific for the C-terminal of SEQ ID NO: 11. In particular, FIGS. 11 and 12 show that there is a region between residues 39 and 53 which is less susceptible to cleavage by endolysosomal enzymes, indicating that this fragment of α-synuclein is resistant to degradation and is more likely to be detected intracellularly in MPS cells. Thus, the function or malfunction of the MPS can be assessed by detecting and measuring the levels of this fragment in MPS cells.

In some embodiments, the method comprises detecting α-synuclein (SEQ ID NO: 10) which has been cleaved between amino acid residues 52 and 53 and/or between residues 72 and 73. In some embodiments, the at least one fragment may be detected by detecting a region of the fragment comprising at least 4 amino acids comprising residue 53, preferably a region comprising residues 53-56, of α-synuclein (SEQ ID NO: 10). In some embodiments, the at least one fragment may be detected by detecting the N-terminal of SEQ ID NO: 12. In some embodiments, the at least one fragment may be detected by detecting a region of the fragment comprising at least 4 amino acids comprising residue 72 of α-synuclein (SEQ ID NO: 10), preferably a region of the fragment comprising residues 69-72 of α-synuclein (SEQ ID NO: 10). In some embodiments, the at least one fragment may be detected by detecting the C-terminal of SEQ ID NO: 12. In some embodiments, the at least one fragment may be detected by detecting a region of the fragment comprising at least 4 amino acids comprising residue 53, preferably comprising residues 53-56, of α-synuclein (SEQ ID NO: 10) and, in addition, detecting a region of the fragment comprising at least 4 amino acids comprising residue 72 of α-synuclein (SEQ ID NO: 10), preferably a region of the fragment comprising residues 69-72 of α-synuclein (SEQ ID NO: 10). In some embodiments, the at least one fragment may be detected by detecting the N-terminal of SEQ ID NO: 12 and detecting a region of the fragment comprising at least 4 amino acids comprising residue 72 of α-synuclein (SEQ ID NO: 10), preferably a region of the fragment comprising residues 69-72 of α-synuclein (SEQ ID NO: 10). In some embodiments, the at least one fragment may be detected by detecting the C-terminal of SEQ ID NO: 12 and detecting a region of the fragment comprising at least 4 amino acids comprising residue 53, preferably a region comprising residues 53-56, of α-synuclein (SEQ ID NO: 10). In some embodiments, the at least one fragment may be detected by detecting the N- and C-terminals of SEQ ID NO: 12. The at least one fragment may be detected using an antibody specific for any of the aforementioned regions or C- or N-terminals. In some embodiments, the at least one fragment may be detected using an antibody specific for a region of the fragment comprising at least 4 amino acids comprising residue 53, preferably comprising residues 53-56, of α-synuclein (SEQ ID NO: 10), or specific for the N-terminal of SEQ ID NO: 12, and an antibody specific for a region of the fragment comprising at least 4 amino acids comprising residue 72 of α-synuclein (SEQ ID NO: 10), preferably a region of the fragment comprising residues 69-72 of α-synuclein (SEQ ID NO: 10), or specific for the C-terminal of SEQ ID NO: 12. In some embodiments, the at least one fragment may be detected using an antibody specific for the N-terminal of SEQ ID NO: 12 and using an antibody specific for a region of the fragment comprising at least 4 amino acids comprising residue 72 of α-synuclein (SEQ ID NO: 10), preferably a region of the fragment comprising residues 69-72 of α-synuclein (SEQ ID NO: 10). In some embodiments, the at least one fragment may be detected using an antibody specific for the C-terminal of SEQ ID NO: 12 and an antibody specific for a region of the fragment comprising at least 4 amino acids comprising residue 53, preferably comprising residues 53-56, of α-synuclein (SEQ ID NO: 10). In some embodiments, the at least one fragment may be detected using an antibody specific for the N-terminal of SEQ ID NO: 12 and an antibody specific for the C-terminal of SEQ ID NO: 12. In particular, FIGS. 11 and 12 show that there is a region between residues 53 and 72 which is less susceptible to cleavage by endolysosomal enzymes, indicating that this fragment of α-synuclein is resistant to degradation and is more likely to be detected intracellularly in MPS cells. Thus, the function or malfunction of the MPS can be assessed by detecting and measuring the levels of this fragment in MPS cells.

In some embodiments, the method comprises detecting α-synuclein (SEQ ID NO: 10) which has been cleaved between amino acid residues 75 and 76 and/or between residues 90 and 91. In some embodiments, the at least one fragment may be detected by detecting a region of the fragment comprising at least 4 amino acids comprising residue 76, preferably a region comprising residues 76-79, of α-synuclein (SEQ ID NO: 10). In some embodiments, the at least one fragment may be detected by detecting the N-terminal of SEQ ID NO: 13. In some embodiments, the at least one fragment may be detected by detecting a region of the fragment comprising at least 4 amino acids comprising residue 90 of α-synuclein (SEQ ID NO: 10), preferably a region of the fragment comprising residues 87-90 of α-synuclein (SEQ ID NO: 10). In some embodiments, the at least one fragment may be detected by detecting the C-terminal of SEQ ID NO: 13. In some embodiments, the at least one fragment may be detected by detecting a region of the fragment comprising at least 4 amino acids comprising residue 76, preferably comprising residues 76-79, of α-synuclein (SEQ ID NO: 10) and, in addition, detecting a region of the fragment comprising at least 4 amino acids comprising residue 90 of α-synuclein (SEQ ID NO: 10), preferably a region of the fragment comprising residues 87-90 of α-synuclein (SEQ ID NO: 10). In some embodiments, the at least one fragment may be detected by detecting the N-terminal of SEQ ID NO: 13 and detecting a region of the fragment comprising at least 4 amino acids comprising residue 90 of α-synuclein (SEQ ID NO: 10), preferably a region of the fragment comprising residues 87-90 of α-synuclein (SEQ ID NO: 10). In some embodiments, the at least one fragment may be detected by detecting the C-terminal of SEQ ID NO: 13 and detecting a region of the fragment comprising at least 4 amino acids comprising residue 76, preferably a region comprising residues 76-79, of α-synuclein (SEQ ID NO: 10). In some embodiments, the at least one fragment may be detected by detecting the N- and C-terminals of SEQ ID NO: 13. The at least one fragment may be detected using an antibody specific for any of the aforementioned regions or C- or N-terminals. In some embodiments, the at least one fragment may be detected using an antibody specific for a region of the fragment comprising at least 4 amino acids comprising residue 76, preferably comprising residues 76-79, of α-synuclein (SEQ ID NO: 10), or specific for the N-terminal of SEQ ID NO: 13, and an antibody specific for a region of the fragment comprising at least 4 amino acids comprising residue 90 of α-synuclein (SEQ ID NO: 10), preferably a region of the fragment comprising residues 87-90 of α-synuclein (SEQ ID NO: 10), or specific for the C-terminal of SEQ ID NO: 13. In some embodiments, the at least one fragment may be detected using an antibody specific for the N-terminal of SEQ ID NO: 13 and using an antibody specific for a region of the fragment comprising at least 4 amino acids comprising residue 90 of α-synuclein (SEQ ID NO: 10), preferably a region of the fragment comprising residues 87-90 of α-synuclein (SEQ ID NO: 10). In some embodiments, the at least one fragment may be detected using an antibody specific for the C-terminal of SEQ ID NO: 13 and an antibody specific for a region of the fragment comprising at least 4 amino acids comprising residue 76, preferably comprising residues 76-79, of α-synuclein (SEQ ID NO: 10). In some embodiments, the at least one fragment may be detected using an antibody specific for the N-terminal of SEQ ID NO: 12 and an antibody specific for the C-terminal of SEQ ID NO: 13. In particular, FIGS. 11 and 12 show that there is a region between residues 76 and 90 which is less susceptible to cleavage by endolysosomal enzymes, indicating that this fragment of α-synuclein is resistant to degradation and is more likely to be detected intracellularly in MPS cells. Thus, the function or malfunction of the MPS can be assessed by detecting and measuring the levels of this fragment in MPS cells.

In some embodiments, more than one fragment of α-synuclein (SEQ ID NO: 10) is detected, for example, two or more of α-synuclein (SEQ ID NO: 10). Each of the two or more fragments of α-synuclein (SEQ ID NO: 10) may be any combination of α-synuclein (SEQ ID NO: 10) which has been cleaved between amino acid residues 38 and 39, and/or between amino acid residues 53 and 54; α-synuclein (SEQ ID NO: 10) which has been cleaved between amino acid residues 52 and 53 and/or between residues 72 and 73; and α-synuclein (SEQ ID NO: 10) which has been cleaved between amino acid residues 75 and 76 and/or between residues 90 and 91. The two or more fragments may be detected by detecting any of the aforementioned regions of the fragment and N- and C-terminals, in any combination. The function or malfunction of the MPS can be better assessed by detecting and measuring the amount of more than one mid-domain fragment of α-synuclein in MPS cells.

Tau

In some embodiments, the disease marker product is Tau (SEQ ID NO: 14), and the method comprises detecting Tau (SEQ ID NO: 14) or at least one fragment thereof. In some embodiments, the detection may be of Tau (SEQ ID NO: 14) which has been cleaved between amino acid residues 305 and 306, and/or between amino acid residues 322 and 323, preferably between residues 280 and 281 and/or between residues 322 and 323. In some embodiments, the at least one fragment may be detected by detecting a region of the fragment comprising at least 4 amino acids comprising residue 306, preferably a region comprising residues 306-309, of Tau (SEQ ID NO: 14). In some embodiments, the at least one fragment is detected by detecting a region of the fragment comprising at least 4 amino acids comprising residue 281, preferably a region comprising residues 281-284, of Tau (SEQ ID NO: 14). In some embodiments, the at least one fragment may be detected by detecting the N-terminal of SEQ ID NO: 15 or 16, preferably the N-terminal of SEQ ID NO: 16. In some embodiments, the at least one fragment may be detected by detecting a region of the fragment comprising at least 4 amino acids comprising residue 322 of Tau (SEQ ID NO: 14), preferably a region of the fragment comprising residues 319-322 of Tau (SEQ ID NO: 14). In some embodiments, the at least one fragment may be detected by detecting the C-terminal of SEQ ID NO: 15 or 16. In some embodiments, the at least one fragment may be detected by detecting a region of the fragment comprising at least 4 amino acids comprising residue 306 or 281, preferably comprising residues 306-309 or 281-284 of Tau (SEQ ID NO: 14) and, in addition, detecting a region of the fragment comprising at least 4 amino acids comprising residue 322 of Tau (SEQ ID NO: 14), preferably a region of the fragment comprising residues 319-322 of Tau (SEQ ID NO: 14). In some embodiments, the at least one fragment may be detected by detecting the N-terminal of SEQ ID NO: 15 or 16 and detecting a region of the fragment comprising at least 4 amino acids comprising residue 322 of Tau (SEQ ID NO: 14), preferably a region comprising residues 319-322 of Tau (SEQ ID NO: 14). In some embodiments, the at least one fragment may be detected by detecting the C-terminal of SEQ ID NO: 15 or 16 and detecting a region of the fragment comprising at least 4 amino acids comprising residue 306 or 281 of Tau (SEQ ID NO: 14), preferably a region of the fragment comprising residues 306-309 or 281-284 of Tau (SEQ ID NO: 14). In some embodiments, the at least one fragment may be detected by detecting the N- and C-terminals of SEQ ID NO: 15 or 16. The at least one fragment may be detected using an antibody specific for any of the aforementioned regions or C- or N-terminals. In some embodiments, the at least one fragment may be detected using an antibody specific for a region of the fragment comprising at least 4 amino acids comprising residue 306 or 281, preferably comprising residues 306-309 or 281-284, of Tau (SEQ ID NO: 14), or specific for the N-terminal of SEQ ID NO: 15 or 16, and an antibody specific for a region of the fragment comprising at least 4 amino acids comprising residue 322 of Tau (SEQ ID NO: 14), preferably a region of the fragment comprising residues 319-322 of Tau (SEQ ID NO: 14), or specific for the C-terminal of SEQ ID NO: 15 or 16. In some embodiments, the at least one fragment may be detected using an antibody specific for the N-terminal of SEQ ID NO: 15 or 16 and using an antibody specific for a region of the fragment comprising at least 4 amino acids comprising residue 322 of Tau (SEQ ID NO: 14), preferably a region of the fragment comprising residues 319-322 of Tau (SEQ ID NO: 14). In some embodiments, the at least one fragment may be detected using an antibody specific for the C-terminal of SEQ ID NO: 15 or 16 and an antibody specific for a region of the fragment comprising at least 4 amino acids comprising residue 306 or 281, preferably comprising residues 306-309 or 281-284, of Tau (SEQ ID NO: 14). In some embodiments, the at least one fragment may be detected using an antibody specific for the N-terminal of SEQ ID NO: 15 or 16 and an antibody specific for the C-terminal of SEQ ID NO: 15 or 16. In particular, it has previously been shown that the R3 region of Tau (residues 306-336) is important in inducing aggregation of Tau, and residues 244-372 of Tau reproduce much of the aggregation behavior of Tau in cells and animal models (Stohr et al., 2017). It has now been found that there is a region between residues 281 and 322 of Tau (SEQ ID NO: 14) which is within this region associated with aggregation and which is less susceptible to cleavage by endolysosomal enzymes (FIG. 13). Thus, it is expected that the function or malfunction of the MPS, and the effect of agents on catabolism and clearance of Tau, can be assessed by measuring and monitoring the catabolism and clearance of this mid-domain fragment of Tau.

TDP-43

In some embodiments, the disease marker product is TDP-43 (SEQ ID NO: 17), and the method comprises detecting TDP-43 (SEQ ID NO: 17) or at least one fragment thereof. In some embodiments, the detection may be of TDP-43 (SEQ ID NO: 17) which has been cleaved between amino acid residues 310 and 311, and/or between amino acid residues 334 and 335. In some embodiments, the at least one fragment may be detected by detecting a region of the fragment comprising at least 4 amino acids comprising residue 311, preferably a region comprising residues 311-314, of TDP-43 (SEQ ID NO: 17). In some embodiments, the at least one fragment may be detected by detecting a region of the fragment comprising at least 4 amino acids comprising residue 334, preferably a region comprising residues 331-334, of TDP-43 (SEQ ID NO: 17). In some embodiments, the at least one fragment may be detected by detecting the N-terminal of SEQ ID NO: 18. In some embodiments, the at least one fragment may be detected by detecting the C-terminal of SEQ ID NO: 18. In some embodiments, the at least one fragment may be detected by detecting a region of the fragment comprising at least 4 amino acids comprising residue 311, preferably comprising residues 311-314, TDP-43 (SEQ ID NO: 17) and, in addition, detecting a region of the fragment comprising at least 4 amino acids comprising residue 334 of TDP-43 (SEQ ID NO: 17), preferably a region of the fragment comprising residues 331-334 of TDP-43 (SEQ ID NO: 17). In some embodiments, the at least one fragment may be detected by detecting the N-terminal of SEQ ID NO: 18 and detecting a region of the fragment comprising at least 4 amino acids comprising residue 334 of TDP-43 (SEQ ID NO: 17), preferably a region comprising residues 331-334 of TDP-43 (SEQ ID NO: 17). In some embodiments, the at least one fragment may be detected by detecting the C-terminal of SEQ ID NO: 18 and detecting a region of the fragment comprising at least 4 amino acids comprising residue 311 of TDP-43 (SEQ ID NO: 17), preferably a region of the fragment comprising residues 311-314 of TDP-43 (SEQ ID NO: 17). In some embodiments, the at least one fragment may be detected by detecting the N- and C-terminals of SEQ ID NO: 18. The at least one fragment may be detected using an antibody specific for any of the aforementioned regions or C- or N-terminals. In some embodiments, the at least one fragment may be detected using an antibody specific for a region of the fragment comprising at least 4 amino acids comprising residue 311, preferably comprising residues 311-314, of TDP-43 (SEQ ID NO: 17), or specific for the N-terminal of SEQ ID NO: 18, and an antibody specific for a region of the fragment comprising at least 4 amino acids comprising residue 334 of TDP-43 (SEQ ID NO: 17), preferably a region of the fragment comprising residues 331-334 of TDP-43 (SEQ ID NO: 17), or specific for the C-terminal of SEQ ID NO: 18. In some embodiments, the at least one fragment may be detected using an antibody specific for the N-terminal of SEQ ID NO: 18 and using an antibody specific for a region of the fragment comprising at least 4 amino acids comprising residue 334 of TDP-43 (SEQ ID NO: 17), preferably a region of the fragment comprising residues 331-334 of TDP-43 (SEQ ID NO: 17). In some embodiments, the at least one fragment may be detected using an antibody specific for the C-terminal of SEQ ID NO: 18 and an antibody specific for a region of the fragment comprising at least 4 amino acids comprising residue 311, preferably comprising residues 311-314, of TDP-43 (SEQ ID NO: 17). In some embodiments, the at least one fragment may be detected using an antibody specific for the N-terminal of SEQ ID NO: 18 and an antibody specific for the C-terminal of SEQ ID NO: 18.

In some embodiments, the detection may be of TDP-43 (SEQ ID NO: 17) which has been cleaved between amino acid residues 245 and 246, and/or between amino acid residues 255 and 256. In some embodiments, the at least one fragment may be detected by detecting a region of the fragment comprising at least 4 amino acids comprising residue 246, preferably a region comprising residues 246-249, of TDP-43 (SEQ ID NO: 17). In some embodiments, the at least one fragment is detected by detecting a region of the fragment comprising at least 4 amino acids comprising residue 255, preferably a region comprising residues 252-255, of TDP-43 (SEQ ID NO: 17). In some embodiments, the at least one fragment may be detected by detecting the N-terminal of SEQ ID NO: 19. In some embodiments, the at least one fragment may be detected by detecting the C-terminal of SEQ ID NO: 18. In some embodiments, the at least one fragment may be detected by detecting a region of the fragment comprising at least 4 amino acids comprising residue 246, preferably comprising residues 246-249, TDP-43 (SEQ ID NO: 17) and, in addition, detecting a region of the fragment comprising at least 4 amino acids comprising residue 255 of TDP-43 (SEQ ID NO: 17), preferably a region of the fragment comprising residues 252-255 of TDP-43 (SEQ ID NO: 17). In some embodiments, the at least one fragment may be detected by detecting the N-terminal of SEQ ID NO: 18 and detecting a region of the fragment comprising at least 4 amino acids comprising residue 255 of TDP-43 (SEQ ID NO: 17), preferably a region comprising residues 252-255 of TDP-43 (SEQ ID NO: 17). In some embodiments, the at least one fragment may be detected by detecting the C-terminal of SEQ ID NO: 18 and detecting a region of the fragment comprising at least 4 amino acids comprising residue 246 of TDP-43 (SEQ ID NO: 17), preferably a region of the fragment comprising residues 246-249 of TDP-43 (SEQ ID NO: 17). In some embodiments, the at least one fragment may be detected by detecting the N- and C-terminals of SEQ ID NO: 18. The at least one fragment may be detected using an antibody specific for any of the aforementioned regions or C- or N-terminals. In some embodiments, the at least one fragment may be detected using an antibody specific for a region of the fragment comprising at least 4 amino acids comprising residue 246, preferably comprising residues 246-249, of TDP-43 (SEQ ID NO: 17), or specific for the N-terminal of SEQ ID NO: 18, and an antibody specific for a region of the fragment comprising at least 4 amino acids comprising residue 255 of TDP-43 (SEQ ID NO: 17), preferably a region of the fragment comprising residues 252-255 of TDP-43 (SEQ ID NO: 17), or specific for the C-terminal of SEQ ID NO: 18. In some embodiments, the at least one fragment may be detected using an antibody specific for the N-terminal of SEQ ID NO: 18 and using an antibody specific for a region of the fragment comprising at least 4 amino acids comprising residue 255 of TDP-43 (SEQ ID NO: 17), preferably a region of the fragment comprising residues 252-255 of TDP-43 (SEQ ID NO: 17). In some embodiments, the at least one fragment may be detected using an antibody specific for the C-terminal of SEQ ID NO: 18 and an antibody specific for a region of the fragment comprising at least 4 amino acids comprising residue 246, preferably comprising residues 246-249, of TDP-43 (SEQ ID NO: 17). In some embodiments, the at least one fragment may be detected using an antibody specific for the N-terminal of SEQ ID NO: 18 and an antibody specific for the C-terminal of SEQ ID NO: 18.

As discussed above, it has been reported that regions of TDP-43, namely residues 246-255, 246-258, 286-331, 311-323 and 311-360, are prone to aggregation (Saini & Chauhan, 2011; Cao et al., 2019). In particular, Saini & Chauhan 2011 showed that a peptide stretching from residue 311 to residue 323, and particularly residues 311-320, inclusive, has an increased tendency for aggregation. However, Saini & Chauhan 2011 did not compare the aggregation of these peptides to peptides which were extended at their C-terminal, and for catalysis, the peptide binding properties are likely to be important for aggregation. Cao et al. 2019 used Cryo-EM to show that peptides extending from residue 312-346 aggregate with tight binding, and showed that the core region of this peptide is residues 312-334. As it has now been found that proteins associated with aggregation and/or toxicity, and abnormal function of the MPS, in neurodegenerative diseases and/or inflammatory activation contain a mid-domain fragment which is prone to aggregation and is resistant to degradation by lysosomal enzymes, it is expected that these aggregation-prone regions of TDP-43 are resistant to degradation by lysosomal enzymes. Thus, it is expected that these mid-domain fragments of TDP-43 can be used to monitor and measure catabolism by the MPS, and to assess the effect of agents on the function or malfunction of the MPS. In addition, in view of the teachings of Saini & Chauhan 2011 and Cao et al. 2019, it is expected that the fragments of TDP-43 associated with aggregation and retained intracellulary in MPS cells, such that they can be used to measure and monitor MPS function or malfunction, are residues 246-255 and residues 311-334 of TDP-43.

MBP

In some embodiments, the disease marker product is MBP (SEQ ID NO: 20), and the method comprises detecting MBP (SEQ ID NO: 20) or at least one fragment thereof. In some embodiments, the detection may be of MBP (SEQ ID NO: 20) which has been cleaved between amino acid residues 29 and 30, and/or between amino acid residues 70 and 71. In some embodiments, the at least one fragment may be detected by detecting a region of the fragment comprising at least 4 amino acids comprising residue 30, preferably a region comprising residues 30-33, of MBP (SEQ ID NO: 20). In some embodiments, the at least one fragment is detected by detecting a region of the fragment comprising at least 4 amino acids comprising residue 70, preferably a region comprising residues 67-70, of MBP (SEQ ID NO: 20). In some embodiments, the at least one fragment may be detected by detecting the N-terminal of SEQ ID NO: 21. In some embodiments, the at least one fragment may be detected by detecting the C-terminal of SEQ ID NO: 21. In some embodiments, the at least one fragment may be detected by detecting a region of the fragment comprising at least 4 amino acids comprising residue 30, preferably comprising residues 30-33 of MBP (SEQ ID NO: 20) and, in addition, detecting a region of the fragment comprising at least 4 amino acids comprising residue 70 of, preferably a region of the fragment comprising residues 67-70 of MBP (SEQ ID NO: 20). In some embodiments, the at least one fragment may be detected by detecting the N-terminal of SEQ ID NO: 21 and detecting a region of the fragment comprising at least 4 amino acids comprising residue 70 of MBP (SEQ ID NO: 20), preferably a region comprising residues 67-70 of MBP (SEQ ID NO: 20). In some embodiments, the at least one fragment may be detected by detecting the C-terminal of SEQ ID NO: 21 and detecting a region of the fragment comprising at least 4 amino acids comprising residue 30 of MBP (SEQ ID NO: 20), preferably a region of the fragment comprising residues 30-33 of MBP (SEQ ID NO: 20). In some embodiments, the at least one fragment may be detected by detecting the N- and C-terminals of SEQ ID NO: 21. The at least one fragment may be detected using an antibody specific for any of the aforementioned regions or C- or N-terminals. In some embodiments, the at least one fragment may be detected using an antibody specific for a region of the fragment comprising at least 4 amino acids comprising residue 30, preferably comprising residues 30-33, of MBP (SEQ ID NO: 20), or specific for the N-terminal of SEQ ID NO: 21, and an antibody specific for a region of the fragment comprising at least 4 amino acids comprising residue 70 of MBP (SEQ ID NO: 20), preferably a region of the fragment comprising residues 67-70 of MBP (SEQ ID NO: 20), or specific for the C-terminal of SEQ ID NO: 21. In some embodiments, the at least one fragment may be detected using an antibody specific for the N-terminal of SEQ ID NO: 21 and using an antibody specific for a region of the fragment comprising at least 4 amino acids comprising residue 70 of MBP (SEQ ID NO: 20), preferably a region of the fragment comprising residues 67-70 of MBP (SEQ ID NO: 20). In some embodiments, the at least one fragment may be detected using an antibody specific for the C-terminal of SEQ ID NO: 21 and an antibody specific for a region of the fragment comprising at least 4 amino acids comprising residue 30, preferably comprising residues 30-33, of MBP (SEQ ID NO: 20). In some embodiments, the at least one fragment may be detected using an antibody specific for the N-terminal of SEQ ID NO: 21 and an antibody specific for the C-terminal of SEQ ID NO: 21. It has been found that there is a region of MBP which is resistant to degradation by endolysosomal enzymes, and this region is between (and includes) amino acid residues 30 and 70 of MBP (FIG. 14). It is known that aggregation of MBP is associated with neurodegenerative diseases, and it is believed that aggregated MBP has neurotoxic effects in Multiple Sclerosis (MS; Frid et al., 2015). As it is known that conditions associated with neurodegeneration and/or inflammatory activation are characterized by a malfunction of the MPS, as discussed above, it is therefore useful to measure and monitor this previously-unknown degradation-resistant fragment of MBP in order to assess the function or malfunction of the MPS. In particular, it has now been found that protein fragments which are associated with aggregation, malfunction of the MPS and neurodegenerative diseases include a region which is resistant to degradation by endolysosomal enzymes and which at least overlaps with a region of the protein which is important for aggregation. It is also expected to be possible to assess the effect of agents on the function or malfunction of the MPS by measuring and monitoring these degradation-resistant mid-domain fragments.

Uses

The method of the invention is useful for assessing and studying MPS function, including on a background of inflammatory activation, as shown by FIG. 8. Thus, the method of the invention finds use is studying MPS malfunction associated with neurodegeneration and/or inflammatory activation. In particular, the method of the present invention allows the study of the effect of inhibiting or activating different parts of the MPS on disease marker product clearance. The Examples show that the precise intracellular action of Aβ clearance can be measured, and that methods for doing so are sensitive enough to measure the effects of proposed therapeutic interventions. These studies also show that Aβ clearance is “druggable”, with strong effects shown by existing drugs and neutraceuticals. It is expected that intracellular clearance of other proteins associated with neurodegenerative diseases and MPS malfunction, such as α-synuclein, Tau, TDP-43 and MBP, can be measured in a similar way.

In some embodiments, the method of the present invention is for identifying new therapeutic interventions. In these embodiments, the effects of one or more candidate agents on MPS function, as measured by disease marker product clearance, can be assessed. Thus, in some embodiments, the method is for screening agents for use in the treatment of neurodegeneration and/or inflammatory activation and/or a condition associated with neurodegeneration. In some embodiments, the method is for screening agents for use in the treatment of Alzheimer's disease, amyloid angiopathy, PD, multiple sclerosis, Frontotemporal Dementia, Amyotrophic Lateral Sclerosis, Lewy Body Dementia, vascular degenerative diseases with inflammatory components, including vascular dementia, Multiple System Atrophy, Progressive Supranuclear Palsy and/or a RNA virus infection. In some embodiments, the method is for screening agents for use in the treatment of Alzheimer's disease and/or amyloid angiopathy.

In some embodiments, the method is for comparing the effects of a panel of different agents on MPS function, by assessing the effects on catalysis of a disease marker product and/or at least one fragment thereof. In some embodiments, the method is for the identification of the agent with the greatest effect on catalysis.

The method of the present invention can also be used for personalised medicine. In such embodiments, the sample of MPS cells and the control MPS cells have been obtained from a human subject diagnosed as having, or suspected of having, neurodegeneration and/or inflammatory activation associated with MPS malfunction. The difference in the intracellular amount of the disease marker product and/or at least one fragment thereof between the sample of MPS cells and the control MPS cells indicates the efficacy of the agent tested in treating the human subject. It is, therefore, particularly advantageous to maintain the sample of MPS cells alive, in order to maintain the MPS function of the human subject. In some embodiments, the method comprises testing a panel of agents on more than one sample of MPS cells and control MPS cells from the human subject, comparing the effects of the different agents on MPS function by detecting the disease marker product and/or at least one fragment thereof in the more than one sample of MPS cells and the control MPS cells, and identifying the agent with the greatest beneficial effect for the human subject. In some embodiments, the method comprises administering the identified agent to the human subject in order to treat the neurodegeneration and/or inflammatory activation.

In some embodiments, the method is for use in diagnosing a neurodegenerative disease and/or inflammatory activation and/or a disease or condition associated with neurodegeneration. In some embodiments, the method is for use in diagnosing Alzheimer's disease, amyloid angiopathy, PD, multiple sclerosis, Frontotemporal Dementia, Amyotrophic Lateral Sclerosis, Lewy Body Dementia, vascular degenerative diseases with inflammatory components, including vascular dementia, Multiple System Atrophy, Progressive Supranuclear Palsy and/or a RNA virus infection. In some embodiments, the method is for use in diagnosing amyloid angiopathy.

In some embodiments, the method is useful for the study of genetic and epigenetic causes of neurodegeneration and/or inflammatory activation. In particular, MPS cells which are maintained alive or cultured retain the genetic and epigenetic changes of their source (such as a particular cell line or human subject).

### Kits

The present invention also relates to a kit comprising an antibody specific for a disease marker product and/or one or more fragments thereof. In some embodiments, the kit comprises more than one antibody, wherein the antibodies are specific for the disease marker fragment and at least one fragment thereof, or the antibodies are specific for different fragments of the disease marker product.

In some embodiments, the kit comprises one or more antibodies for the detection of any one of the above-described regions, or N- or C-terminals, of Aβ₁₋₄₂ (SEQ ID NO: 1) or Aβ₁₋₄₀ (SEQ ID NO: 2), and/or a fragment thereof. In some embodiments, the kit comprises an antibody specific for a region of a fragment of Aβ₁₋₄₂ (SEQ ID NO: 1) or Aβ₁₋₄₀ (SEQ ID NO: 2) comprising at least 4 amino acids comprising residue 21, 34 or 40 of Aβ₁₋₄₂ (SEQ ID NO: 1) or Aβ₁₋₄₀ (SEQ ID NO: 2). In some embodiments, the kit comprises an antibody specific for a region of a fragment of Aβ₁₋₄₂ (SEQ ID NO: 1) or Aβ₁₋₄₀ (SEQ ID NO: 2) comprising residues 21-24, 31-34 or 39-40 of Aβ₁₋₄₂ (SEQ ID NO: 1) or Aβ₁₋₄₀ (SEQ ID NO: 2). In some embodiments, the kit comprises an antibody specific for a region of a fragment of Aβ₁₋₄₂ (SEQ ID NO: 1) or Aβ₁₋₄₀ (SEQ ID NO: 2) comprising at least 4 amino acids comprising residue 21, preferably specific for a region of the fragment comprising residues 21-24, of Aβ₁₋₄₂ (SEQ ID NO: 1) or Aβ₁₋₄₀ (SEQ ID NO: 2), and an antibody specific for a region of a fragment of Aβ₁₋₄₂ (SEQ ID NO: 1) or Aβ₁₋₄₀ (SEQ ID NO: 2) comprising at least 4 amino acids comprising residue 34, preferably specific for a region of a fragment comprising residues 31-34, of Aβ₁₋₄₂ (SEQ ID NO: 1) or Aβ₁₋₄₀ (SEQ ID NO: 2). In some embodiments, the kit comprises an antibody specific for a region of a fragment of Aβ₁₋₄₂ (SEQ ID NO: 1) or Aβ₁₋₄₀ (SEQ ID NO: 2) comprising at least 4 amino acids comprising residue 21, preferably specific for a region of the fragment comprising residues 21-24, of Aβ₁₋₄₂ (SEQ ID NO: 1) or Aβ₁₋₄₀ (SEQ ID NO: 2), and an antibody specific for a region of a fragment of Aβ₁₋₄₂ (SEQ ID NO: 1) or Aβ₁₋₄₀ (SEQ ID NO: 2) comprising at least 4 amino acids comprising residue 40, preferably specific for a region of a fragment comprising residues 39-40, of Aβ₁₋₄₂ (SEQ ID NO: 1) or Aβ₁₋₄₀ (SEQ ID NO: 2). In some embodiments, the kit comprises an antibody specific for the N-terminal of SEQ ID NO: 6 and an antibody specific for the C-terminal of SEQ ID NO: 6; or an antibody specific for the N-terminal of SEQ ID NO: 6 and an antibody specific for the C-terminal of SEQ ID NO: 1 or SEQ ID NO: 2. In some embodiments, the kit comprises an antibody specific for the N-terminal of SEQ ID NO: 6 and an antibody specific for a region of a fragment of Aβ₁₋₄₂ (SEQ ID NO: 1) or Aβ₁₋₄₀ (SEQ ID NO: 2) comprising residues 39-40 of Aβ₁₋₄₂ (SEQ ID NO: 1) or Aβ₁₋₄₀ (SEQ ID NO: 2). In some embodiments, the kit comprises an antibody specific for a region of a fragment of Aβ₁₋₄₂ (SEQ ID NO: 1) or Aβ₁₋₄₀ (SEQ ID NO: 2) comprising residues 21-24 of Aβ₁₋₄₂ (SEQ ID NO: 1) or Aβ₁₋₄₀ (SEQ ID NO: 2) and an antibody specific for the C-terminal of SEQ ID NO: 6. In some embodiments, the kit comprises an antibody specific for a fragment of Aβ₁₋₄₂ (SEQ ID NO: 1) or Aβ₁₋₄₀ (SEQ ID NO: 2) comprising residues 21-24 of Aβ₁₋₄₂ (SEQ ID NO: 1) or Aβ₁₋₄₀ (SEQ ID NO: 2) and an antibody specific for the C-terminus of Aβ₁₋₄₂ (SEQ ID NO: 1) or Aβ₁₋₄₀ (SEQ ID NO: 2). In some embodiments, the kit comprises antibodies specific for each of the aforementioned regions, N-terminals and C-terminals, for example, an antibody specific for a region of a fragment of Aβ₁₋₄₂ (SEQ ID NO: 1) or Aβ₁₋₄₀ (SEQ ID NO: 2) comprising at least 4 amino acids comprising residue 21 of Aβ₁₋₄₂ (SEQ ID NO: 1) or Aβ₁₋₄₀ (SEQ ID NO: 2), an antibody specific for a region of a fragment of Aβ₁₋₄₂ (SEQ ID NO: 1) or Aβ₁₋₄₀ (SEQ ID NO: 2) comprising at least 4 amino acids comprising residue 34 of Aβ₁₋₄₂ (SEQ ID NO: 1) or Aβ₁₋₄₀ (SEQ ID NO: 2) and an antibody specific for a region of a fragment of Aβ₁₋₄₂ (SEQ ID NO: 1) or Aβ₁₋₄₀ (SEQ ID NO: 2) comprising at least 4 amino acids comprising residue 40 of Tau Aβ₁₋₄₂ (SEQ ID NO: 1) or Aβ₁₋₄₀ (SEQ ID NO: 2).

In some embodiments, the kit comprises one of an antibody raised against SEQ ID NO: 3, an antibody raised against SEQ ID NO: 4 and an antibody raised against SEQ ID NO: 5, an antibody raised against SEQ ID NO: 7, an antibody raised against SEQ ID NO: 8 and an antibody raised against SEQ ID NO: 9. In some embodiments, the kit comprises an antibody raised against SEQ ID NO: 7 and an antibody raised against SEQ ID NO: 4, 5, 8 or 9. In some embodiments, the kit comprises an antibody raised against SEQ ID NO: 7 and an antibody raised against SEQ ID NO: 5 or 8. In some embodiments, the kit comprises an antibody raised against SEQ ID ON: 7 and an antibody raised against SEQ ID NO: 4. In some embodiments, the kit comprises an antibody raised against SEQ ID NO: 3 and an antibody raised against SEQ ID NO: 9. In some embodiments the kit comprises an antibody raised against SEQ ID NO: 2 and an antibody raised against SEQ ID NO: 4, an antibody raised against SEQ ID NO: 2 and an antibody raised against SEQ ID NO: 5, 8 or 9, or an antibody raised against SEQ ID NO: 4 and an antibody raised against SEQ ID NO: 5, 8 or 9. Preferably, the kit comprises an antibody raised against SEQ ID NO: 3, an antibody raised against SEQ ID NO: 4 and an antibody raised against SEQ ID NO: 5. In some embodiments, the kit comprises an antibody raised against SEQ ID NO: 7 and an antibody specific for a region of a fragment of Aβ₁₋₄₂ (SEQ ID NO: 1) and Aβ₁₋₄₀ (SEQ ID NO: 2) comprising at least 4 amino acids comprising residue 34 of Aβ₁₋₄₂ (SEQ ID NO: 1) and Aβ₁₋₄₀ (SEQ ID NO: 2); an antibody raised against SEQ ID NO: 7 and an antibody raised against SEQ ID NO: 8; or an antibody raised against SEQ ID NO: 9 and an antibody specific for the N-terminal of SEQ ID NO: 6. In some embodiments, the kit comprises an antibody raised against SEQ ID NO: 7, an antibody raised against SEQ ID NO: 8, an antibody raised against SEQ ID NO: 9, an antibody specific for a region of a fragment of Aβ₁₋₄₂ (SEQ ID NO: 1) and Aβ₁₋₄₀ (SEQ ID NO: 2) comprising at least 4 amino acids comprising residue 34 of Aβ₁₋₄₂ (SEQ ID NO: 1) and Aβ₁₋₄₀ (SEQ ID NO: 2) and an antibody specific for the N-terminal of SEQ ID NO: 6. In some embodiments, the kit comprises an antibody raised against SEQ ID NO: 3, an antibody raised against SEQ ID NO: 4, an antibody raised against SEQ ID NO: 7, an antibody raised against SEQ ID NO: 8 and an antibody raised against SEQ ID NO: 9. In some embodiments, the kit comprises one or more antibodies for the detection of any one of the above-described regions, or N- or C-terminals, of α-synuclein and/or a fragment thereof. In some embodiments, the kit comprises an antibody specific for a region of a fragment of α-synuclein (SEQ ID NO: 10) comprising at least 4 amino acids comprising residue 39, 53, 72, 76 or 90 of α-synuclein (SEQ ID NO: 10). In some embodiments, the kit comprises an antibody specific for a region of a fragment of α-synuclein (SEQ ID NO: 10) comprising residues 39-42, 50-53, 53-56, 69-72, 76-79 or 87-90 of α-synuclein (SEQ ID NO: 10). In some embodiments, the kit comprises an antibody specific for a region of a fragment of α-synuclein (SEQ ID NO: 10) comprising at least 4 amino acids comprising residue 39, preferably specific for a region of the fragment comprising residues 39-42, of α-synuclein (SEQ ID NO: 10), and an antibody specific for a region of a fragment of α-synuclein (SEQ ID NO: 10) comprising at least 4 amino acids comprising residue 53, preferably specific for a region of a fragment comprising residues 50-43, of α-synuclein (SEQ ID NO: 10). In some embodiments, the kit comprises an antibody specific for a region of a fragment of α-synuclein (SEQ ID NO: 10) comprising at least 4 amino acids comprising residue 53, preferably specific for a region of the fragment comprising residues 53-56, of α-synuclein (SEQ ID NO: 10), and an antibody specific for a region of a fragment of α-synuclein (SEQ ID NO: 10) comprising at least 4 amino acids comprising residue 72, preferably specific for a region of a fragment comprising residues 69-72, of α-synuclein (SEQ ID NO: 10). In some embodiments, the kit comprises an antibody specific for a region of a fragment of α-synuclein (SEQ ID NO: 10) comprising at least 4 amino acids comprising residue 76, preferably specific for a region of the fragment comprising residues 76-79, of α-synuclein (SEQ ID NO: 10), and an antibody specific for a region of a fragment of α-synuclein (SEQ ID NO: 10) comprising at least 4 amino acids comprising residue 90, preferably specific for a region of a fragment comprising residues 87-90, of α-synuclein (SEQ ID NO: 10). In some embodiments, the kit comprises an antibody specific for the N-terminal of SEQ ID NO: 11 and an antibody specific for the C-terminal of SEQ ID NO: 11; or an antibody specific for the N-terminal of SEQ ID NO: 12 and an antibody specific for the C-terminal of SEQ ID NO: 12; or an antibody specific for the N-terminal of SEQ ID NO: 13 and an antibody specific for the C-terminal of SEQ ID NO: 13. In some embodiments, the kit comprises antibodies specific for each of the aforementioned regions, N-terminals and C-terminals, for example, an antibody specific for a region of a fragment of α-synuclein (SEQ ID NO: 10) comprising at least 4 amino acids comprising residue 39 of α-synuclein (SEQ ID NO: 10), an antibody specific for a region of a fragment of α-synuclein (SEQ ID NO: 10) comprising at least 4 amino acids comprising residue 53 of α-synuclein (SEQ ID NO: 10), an antibody specific for a region of a fragment of α-synuclein (SEQ ID NO: 10) comprising at least 4 amino acids comprising residue 72 of α-synuclein (SEQ ID NO: 10), an antibody specific for a region of a fragment of α-synuclein (SEQ ID NO: 10) comprising at least 4 amino acids comprising residue 76 of α-synuclein (SEQ ID NO: 10) and an antibody specific for a region of a fragment of α-synuclein (SEQ ID NO: 10) comprising at least 4 amino acids comprising residue 90 of α-synuclein (SEQ ID NO: 10). In these embodiments, the kit may comprise an antibody specific for a first region of a fragment of α-synuclein (SEQ ID NO: 10) comprising at least 4 amino acids comprising residue 53 of α-synuclein (SEQ ID NO: 10) and an antibody specific for a second region of a fragment of α-synuclein (SEQ ID NO: 10) comprising at least 4 amino acids comprising residue 53 of α-synuclein (SEQ ID NO: 10), wherein the first region is different from the second region.

In some embodiments, the kit comprises one or more antibodies for the detection of any one of the above-described regions, or N- or C-terminals, of Tau (SEQ ID NO: 14), and/or a fragment thereof. In some embodiments, the kit comprises an antibody specific for a region of a fragment of Tau (SEQ ID NO: 14) comprising at least 4 amino acids comprising residue 281, 306, 322 of Tau (SEQ ID NO: 14). In some embodiments, the kit comprises an antibody specific for a region of a fragment of Tau (SEQ ID NO: 14) comprising residues 281-284, 306-309 or 319-322 of Tau (SEQ ID NO: 14). In some embodiments, the kit comprises an antibody specific for a region of a fragment of Tau (SEQ ID NO: 14) comprising at least 4 amino acids comprising residue 281, preferably specific for a region of the fragment comprising residues 281-284, of Tau (SEQ ID NO: 14), and an antibody specific for a region of a fragment of Tau (SEQ ID NO: 14) comprising at least 4 amino acids comprising residue 322, preferably specific for a region of a fragment comprising residues 319-322, of Tau (SEQ ID NO: 14). In some embodiments, the kit comprises an antibody specific for a region of a fragment of Tau (SEQ ID NO: 14) comprising at least 4 amino acids comprising residue 306, preferably specific for a region of the fragment comprising residues 306-309, of Tau (SEQ ID NO: 14), and an antibody specific for a region of a fragment of Tau (SEQ ID NO: 14) comprising at least 4 amino acids comprising residue 322, preferably specific for a region of a fragment comprising residues 319-322, of Tau (SEQ ID NO: 14). In some embodiments, the kit comprises an antibody specific for the N-terminal of SEQ ID NO: 15 and an antibody specific for the C-terminal of SEQ ID NO: 15; or an antibody specific for the N-terminal of SEQ ID NO: 16 and an antibody specific for the C-terminal of SEQ ID NO: 16. In some embodiments, the kit comprises antibodies specific for each of the aforementioned regions, N-terminals and C-terminals, for example, an antibody specific for a region of a fragment of Tau (SEQ ID NO: 14) comprising at least 4 amino acids comprising residue 218 of Tau (SEQ ID NO: 14), an antibody specific for a region of a fragment of Tau (SEQ ID NO: 14) comprising at least 4 amino acids comprising residue 305 of Tau (SEQ ID NO: 14) and an antibody specific for a region of a fragment of Tau (SEQ ID NO: 14) comprising at least 4 amino acids comprising residue 322 of Tau (SEQ ID NO: 14).

In some embodiments, the kit comprises one or more antibodies for the detection of any one of the above-described regions, or N- or C-terminals, of TDP-43 (SEQ ID NO: 17), and/or a fragment thereof. In some embodiments, the kit comprises an antibody specific for a region of a fragment of TDP-43 (SEQ ID NO: 17) comprising at least 4 amino acids comprising residue 246, 255, 311 or 334 of TDP-43 (SEQ ID NO: 17). In some embodiments, the kit comprises an antibody specific for a region of a fragment of TDP-43 (SEQ ID NO: 17) comprising residues 246-249, 252-255, 311-314 or 331-334 of TDP-43 (SEQ ID NO: 17). In some embodiments, the kit comprises an antibody specific for a region of a fragment of TDP-43 (SEQ ID NO: 17) comprising at least 4 amino acids comprising residue 246, preferably specific for a region of the fragment comprising residues 246-249, of TDP-43 (SEQ ID NO: 17), and an antibody specific for a region of a fragment of TDP-43 (SEQ ID NO: 17) comprising at least 4 amino acids comprising residue 255, preferably specific for a region of a fragment comprising residues 252-255, of TDP-43 (SEQ ID NO: 17). In some embodiments, the kit comprises an antibody specific for a region of a fragment of TDP-43 (SEQ ID NO: 17) comprising at least 4 amino acids comprising residue 311, preferably specific for a region of the fragment comprising residues 311-314, of TDP-43 (SEQ ID NO: 17), and an antibody specific for a region of a fragment of TDP-43 (SEQ ID NO: 17) comprising at least 4 amino acids comprising residue 334, preferably specific for a region of a fragment comprising residues 331-334, of TDP-43 (SEQ ID NO: 17). In some embodiments, the kit comprises an antibody specific for the N-terminal of SEQ ID NO: 18 and an antibody specific for the C-terminal of SEQ ID NO: 18; or an antibody specific for the N-terminal of SEQ ID NO: 19 and an antibody specific for the C-terminal of SEQ ID NO: 19. In some embodiments, the kit comprises antibodies specific for each of the aforementioned regions, N-terminals and C-terminals, for example, an antibody specific for a region of a fragment of TDP-43 (SEQ ID NO: 17) comprising at least 4 amino acids comprising residue 311 of TDP-43 (SEQ ID NO: 17), an antibody specific for a region of a fragment of TDP-43 (SEQ ID NO: 17) comprising at least 4 amino acids comprising residue 334 of TDP-43 (SEQ ID NO: 17), an antibody specific for a region of a fragment of TDP-43 (SEQ ID NO: 17) comprising at least 4 amino acid residues comprising residue 246 of TDP-43 (SEQ ID NO: 17) and an antibody specific for a region of a fragment of TDP-43 (SEQ ID NO: 17) comprising at least 4 amino acids comprising residue 255 of TDP-43 (SEQ ID NO: 17).

In some embodiments, the kit comprises one or more antibodies for the detection of any one of the above-described regions, or N- or C-terminals, of MBP (SEQ ID NO: 20), and/or a fragment thereof. In some embodiments, the kit comprises an antibody specific for a region of a fragment of MBP (SEQ ID NO: 20) comprising at least 4 amino acids comprising residue 30 or 70 of MBP (SEQ ID NO: 20). In some embodiments, the kit comprises an antibody specific for a region of a fragment of MBP (SEQ ID NO: 20) comprising residues 30-33 or 67-70 of MBP (SEQ ID NO: 20). In some embodiments, the kit comprises an antibody specific for a region of a fragment of MBP (SEQ ID NO: 20) comprising at least 4 amino acids comprising residue 30, preferably specific for a region of the fragment comprising residues 30-33, of MBP (SEQ ID NO: 20), and an antibody specific for a region of a fragment of MBP (SEQ ID NO: 20) comprising at least 4 amino acids comprising residue 70, preferably specific for a region of a fragment comprising residues 67-70, of MBP (SEQ ID NO: 20). In some embodiments, the kit comprises an antibody specific for the N-terminal of SEQ ID NO: 21 and an antibody specific for the C-terminal of SEQ ID NO: 21.

In some embodiments, the kit comprises antibodies for detecting fragments of more than one protein. In these embodiments, the kit may comprise any combination of the above-described antibodies.

The kits may be used in the methods of the present invention, as disclosed above.

### EXAMPLES

### Statistics (Examples 1-4)

To adjust for inter-day variations in measurements for the different experiments, values were normalized using the baseline condition as the reference for each day of experiments using the following formula: [(biomarker value/mean biomarker value)/100)].

Statistical analyses were carried out with one-way ANOVA models. If a significant (p<0.05) omnibus (F-test) ANOVA was shown, planned comparisons according to our hypotheses were carried out. This included a main comparison between the reference condition and experimental conditions (endolysosomal inhibitors and autophagosome activators). The Figures thus indicate within each bar the percentage change, and associated statistical significance (p-value) or lack thereof (n.s.), as compared to the reference condition (100%) (i.e. the main comparison). For some experiments (FIGS. 4, 5 and 7), additional comparisons between experimental conditions were conducted in accordance with hypotheses. Significant between-group differences for these comparisons are shown with horizontal bars with the associated statistical significance (p-value), or lack thereof (n.s.).

### Example 1

Aβ catabolism and detoxification, and the effect of example inhibitors and activators thereon, was measured using antibodies specific for intracellular Aβ mid-domain peptides, i.e. catabolism products. In particular, inhibition of endolysosomal activity is expected to reduce degradation and increase concentrations of species, here studied with Bafilomycin, which blocks V-ATPase and inhibits acidification of endolysosomes, thereby inhibiting endolysosomal enzyme activities. Addition of inhibitors of endolysosomal enzymes Neprilysin (Saido 2013) and Insulin Degradation Enzymes are expected to have similar effects (Jha, Jha et al. 2015, Sikanyika, Parkington et al. 2019), whereas BACE1 is involved both in Aβ generation and 34′-catalysis (Vassar, Kovacs et al. 2009, Liebsch, Kulic et al. 2019). Enhancing macroautophagy using Rapamycin is also expected to increase Aβ catabolism (FIG. 3; Spilman, Podlutskaya et al. 2010, Malik, Maddison et al. 2019)

Antibodies and Primary Assays

Immunoassays were developed on the single molecule array (Simoa) platform (Quanterix, US) using proprietary antibodies directed against different epitopes of the amyloid β protein (Shown in FIG. 1; SEQ ID NOs: 3-5). The Aβ_{x-40/42} assay used a 40-42 end-specific sheep monoclonal AB.4D7 (Bioventix plc, UK) as the capture antibody, and a sheep monoclonal AB.2A9 (Bioventix plc, UK) with a mid-domain epitope (SEQ ID NO: 7) as the detection antibody. The 40-42 end-specific antibody was raised against the epitope shown in SEQ ID NO: 5, and binds the C-terminal of both Aβ₁₋₄₂ (SEQ ID NO: 1) and Aβ₁₋₄₀ (SEQ ID NO: 2). The Aβ₂₀₋ₓ assay used the same AB.4D7 antibody as the capture antibody, and the highly specific 20-cut point sheep monoclonal antibody, ABNT.2B8 (Bioventix plc, UK) as the detection antibody. For the Aβₓ₋₃₄ assay, AB.2A9 was used as the capture antibody and an anti-complex antibody, BVX.33420/H3 (Bioventix plc, UK), which preferentially binds a fragment having the C-terminal of SEQ ID NO: 6, was used as the detection antibody, ensuring mid-domain x-34 specificity.

Reagents

Amyloid Beta (Aβ) peptide (1-40) human, cat no A1124 (ApexBio, US). Bafilomycin A1, Cat no B1793 (Sigma, US). Rapamycin, Cat no R0395 (Sigma, US). Neprilysin inhibitor; Phosphoramidon disodium salt, Cat no 6333 (Tocris, UK). BACE inhibitor; LY2886721, Cat no 7081 (Tocris, UK). Insulin degrading enzyme inhibitor; 6bK, Cat no 5402 (Tocris, UK).

Chase Assays THP-1 cells were differentiated toward macrophages by a 48 hour treatment with 100 nM TPA (12-O-tetradecanoylphorbol-13-acetate). Media was replaced with fresh medium with or without modulators of the autophagic/endolysosomal system: 100 nM Bafilomycin, 100 nM Rapamycin, 100 μM Neprilysin inhibitor, 200 nM BACE inhibitor, or 1 μM IDE inhibitor, or a triple combination of the enzyme inhibitors Neprilysin inhibitor, BACE inhibitor and IDE inhibitor. The cells were incubated for 1 hour before adding 100 ng/ml Abeta40 to the cells. Cells were further incubated for 2 hours before removing Abeta40 and a continued 4 hours chase with or without the respective modulators. Cells were washed before adding lysis buffer. Lysates were kept at −80° C. until analysis.

Results

The results of these assays are shown in FIGS. 4 and 5. In particular, FIGS. 4 and 5 show that Aβ_{x-40/42} and Aβ₂₀₋ₓ are significantly reduced during 4 hours chase, whereas Aβₓ₋₃₄ is unchanged. The results also show that endolysosmal inhibition (Bafilomycin) significantly increases Aβ_{x-40/42} and Aβ₂₀₋ₓ, and non-significantly increases Aβₓ₋₃₄, and that enhanced autophagy using Rapamycin leaves Aβ_{x-40/42} and Aβ₂₀₋ₓ levels unchanged, but strongly reduces Aβₓ₋₃₄ levels. FIG. 5 shows that, in comparison to 4 hour chase, the triple combination of enzyme inhibitors (Neprilysin inhibitor, BACE inhibitor and IDE inhibitor) significantly increases Aβ_{x-40/42} levels, but does not affect the levels of Aβ₂₀₋ₓ, and results in a non-significant increase (trend levels) of Aβₓ₋₃₄ (few experiments).

Inhibition using only Neprilysin inhibitor increases Aβ_{x-40/42} and Aβ₂₀₋ₓ (FIGS. 5C and 5A, respectively), whereas Aβₓ₋₃₄ levels are unchanged. Inhibition using only BACE inhibitor increases Aβ₂₀₋ₓ, but has no effect on Aβₓ₋₃₄ or Aβ_{x-40/42}. Inhibition using IDE inhibitor increases Aβ₂₀₋ₓ, but has no effect on levels of either Aβₓ₋₃₄ or Aβ_{x-40/42}.

Thus, the effect of different MPS inhibitors and activators on MPS function, and different specific points of the MPS, can be assessed. The method is sufficiently sensitive to measure the effects of different inhibitors and activators.

### Example 2

The effects of proposed therapeutic interventions for AD (namely, a FFAR4 agonist and an α7 nAChR PAM; WO 2020/2020/212627) on Aβ catabolism and detoxification, as an indication of MPS function, were also assessed.

Method

An immunoassay was set up on a Mesoscale platform using the same antibodies as, and a similar method to, Example 1. Cells were incubated overnight (16 hours) with DHA (100 μM) and/or NS1738 (10 μM). Aβ₁₋₄₀ was added at a concentration of 500 ng/ml, and removed from the media before the 4 hour chase. DHA and NS1738 were removed from the media during the 4 hour chase.

Results

FIG. 6 shows that the combination of DHA+NS1738 significantly decreases all three of Aβ₂₀₋ₓ, Aβₓ₋₃₄ and Aβₓ₋₄₀, and that each of NS1738 alone and DHA alone decreased Aβₓ₋₃₄. In this Example, the reference condition for the main comparison was Aβ40 4 hour chase.

Thus, the effects of different therapeutic interventions for AD on MPS function, and specific points thereof, can be assessed by measuring the effects of the therapeutic interventions on different catalysis products.

### Example 3

The effect of the combination of DHA and NS1738 on MPS function was compared to the effects of the Rapamycin and Bafilomycin. The immunoassay methods were the same as for Example 2, and the results are shown in FIG. 7.

FIG. 7 shows that the combination of DHA and NS1738 significantly reduced levels of all three β fragments, showing that this drug combination is effective in improving MPS function.

### Example 4

As AD is associated with inflammatory activation, the effect of different drugs and drug combinations on MPS function and Aβ clearance on a background of inflammatory activation was assessed.

Method

The immunoassay was set up on a Mesoscale platform. THP-1 cells were differentiated towards phagocytic monocytes/macrophages during a 48 h incubation with 12-O-tetra-decanoylphorbol-13-acetate (TPA). Media were replaced with fresh medium containing LPS alone or LPS together with DHA or NS1738 or combination of DHA and NS1738, for an overnight incubation (16 h). The marker protein (Aβ₁₋₄₀) was added to the cells for an additional 2 h incubation. Media were replaced with fresh medium without any additives and the cells were further incubated for a 4 h chase period. The cells were kept cold, media removed, and the cells washed with cold PBS. Lysis buffer containing protease inhibitors and sodium dodecyl sulfate (SDS) was added to the cells. After lysis, the cells were scraped and transferred to tubes and kept in a −80° C. freezer. Lysates were sonicated and treated with DNase before analysis on the degradation assays.

Results

FIG. 8 shows the results of the clearance assays, and shows that all of the drugs and combinations thereof significantly reduce all three Aβ fragments. For this experiment, the reference condition for the main comparison was LPS+Aβ40 4 hour chase.

Thus, the clearance assay can be used to model the inflammatory background associated with neurodegenerative diseases, providing a more accurate assessment of the in vivo effects of therapeutic interventions. In addition, the interaction between inflammatory activation and MPS function can be assessed.

Of note is the prominent differences in the effects of endolysosomal enzyme inhibitors on catalysis of Aβ₂₀₋ₓ and Aβₓ₋₄₀ epitopes, as compared to Aβₓ₋₃₄ epitopes (FIGS. 5A and 5C vs. FIG. 5B). However, treatment with DHA and NS were similarly effective for all fragments (FIGS. 7 and 8). These differences likely arise from engagement of different enzymes for catalysis, where DHA and NS likely counteract inflammation (Aβ- or LPS-induced) and engage the constitutive Cathepsin proteases (Creasy & McCoy 2011). The Aβₓ₋₃₄ assay thus stands out, producing a likely broadly amenable marker for Cathepsin engagement and modulation (Hook et al., 2002, Cermak et al., 2016).

### Example 5

The Aβ clearance assay was verified using monocytes obtained from blood donors.

Methods

The immunoassay, developed on the Quanterix (CA, USA) Single Molecule Array (Simoa) platform, used a proprietary anti-mid-domain sheep monoclonal antibody (mAb 2A9) as the capture antibody (disclosed in U.S. Pat. No. 9,625,474). The detector reagent was an anti-complex antibody (Bio-Rad HuCAL technology) aiding an optimal Aβ mid-domain peptide specificity and assay sensitivity (CTAD poster by Torsetnes et al. 2019). These are shown in FIG. 9. The anti-complex antibody binds only if both Ab2A9 and a 34-ending mid-domain AB peptide is present (Torsetnes et al., 2019)

Monocytes from blood donors were isolated from the leukocyte fraction using a combination of density gradient centrifugation and RosetteSep antibodies from StemCell Technologies (Canada). A monocyte count was performed before cell lysis, and the monocyte concentration was adjusted to 20,000 cells/μl.

33 samples from healthy donors (18 females and 15 males) were age-matched, and spanned a range of 20-70 years.

Results

The assay, with the two anti-Aβ mid-domain antibodies analysed in the Simoa platform, gave a sensitive immunoassay detecting Aβ mid-domain peptides with a limit of detection (LOD) of 0.78 pg/ml, and a lower limit of quantification (LLOQ) of 1.38 pg/ml (LOD and LLOQ were evaluated by the Simoa 4PL Assay development tool), resulting in measurable levels of the peptide in monocyte lysates (FIG. 10 and Table 1).

TABLE 1

| Samples | Mean conc pg/ml | Mean CV % |
|---|---|---|
| Monocyte lysates | 8.5 (range 4.4-17.0) | 7.9 (range 0.5-20.8) |

Of the 33 samples, 1 was below LOD and one was below LLOQ.

Dilution fact and spike, and recovery in monocyte lysates: A pooled sample was aliquoted and tested for dilution effect, as well as the effect of adding different amounts of a target mid-domain Aβ peptide. The recovery results are shown in Table 2.

TABLE 2

| | | | | Fiited | Corrected | |
|---|---|---|---|---|---|---|
| | Dilution | Spiked with | | conc, | fitted conc, | Recovery |
| Sample | Factor | Aβ₂₀₋₃₄ | | pg/ml | pg/ml | % |
| Monocyte | 1:2 | n/a | | 8.0 | 16.0 | 100% |
| lysate pool | | | | | | |
| Monocyte | 1:4 | n/a | | 4.3 | 17.2 | 108% |
| lysate pool | | | | | | |
| Monocyte | 1:2 | 3 | pg/ml | 10.1 | n/a | 92% |
| lysate pool | | | | | | |
| Monocyte | 1:2 | 6 | pg/ml | 12.7 | n/a | 91% |
| lysate pool | | | | | | |
| Monocyte | 1:2 | 12 | pg/ml | 15.5 | n/a | 78% |
| lysate pool | | | | | | |

Depletion studies: A pooled sample was aliquoted and a target mid-domain Aβ peptide was added to half of the samples. An unspecific antibody (LB509) and the capture antibody (mAb 2A9) were separately pre-incubated in both treated and untreated samples, with the antibodies being added in a ratio of 1:3 compared to the amount of capture antibody used in the determination step used to produce Table 2. The results of this depletion study are shown in Table 3.

TABLE 3

| | | | Unspecific Ab | | mAb 2A9 | |
|---|---|---|---|---|---|---|
| | Untreated | | depletion (LB509) | | depletion | |
| Monocyte lysate pool | 6.4 | pg/ml | 6.7 | pg/ml | 0 | pg/ml |
| Percent of untreated | 100% | | 100% | | 0% | |
| Monocyte lysate pool + | 13.8 | pg/ml | 11.1 | pg/ml | 0 | pg/ml |
| 12 pg/ml Aβ₂₀₋₃₄ | | | | | | |

| | | | |
|---|---|---|---|
| Percent of untreated | 100% | 80% | 0% |

Thus, the immunoassay quantitated Aβ mid-domain peptides in monocytes from healthy blood donors and demonstrated reliable results through good recovery in spiking dilution studies (Table 2). In addition, the depletion study with excess antibodies removed the signals (Table 3). This shows that the immunoassay is able to detect intracellular Aβ, has acceptable specificity in biological samples and is highly sensitive.

### Example 6

α-Synuclein, Tau and MBP were enzymatically degraded using enzymes known to be involved in intracellular and extracellular protein degradation, and the digestion products were characterised.

α-Synuclein was digested with five enzymes known to be involved in intracellular end extracellular protein degradation; Cathepsin B (CatB), Cathepsin D (CatD), Endothelin Converting Enzyme-1 (ECE-1), Insulin degrading enzyme (IDE) and Neprilysin (NEP).

Tau and MBP were digested with four enzymes known to be involved in intracellular end extracellular protein degradation; Cathepsin B (CatB), Cathepsin D (CatD), Endothelin Converting Enzyme-1 (ECE-1) and Insulin degrading enzyme (IDE).

The peptide hydrolysis products were identified by LC-MS. The enzymatic digestion products were characterized after different incubation times, thus both the initial sites of cleavage and a full overview of all peptides produced at longer digestion times were identified.

Material α-Synuclein (cat no S-1001-2, rPeptide, Georgia, USA) was dissolved in 1 ml phosphate buffered saline (PBS) to a concentration of 1 mg/ml, aliquoted and stored at −20° C. until use.

Tau-441, 2N4R (cat no T-1001-2, rPeptide, Georgia, USA) was dissolved in 1 ml water to a concentration of 1 mg/ml, aliquoted and stored at −20° C. until use.

Myelin basic protein (MBP), 1-197 aa, His-tagged (cat no PAT-82347-2, Nordic Biosite) supplied at a concentration of 0.5 mg/ml, aliquoted and stored at −20° C. until use.

CatD (human liver, product number C8696, Sigma-Aldrich, Saint Louis, MO, USA) was dissolved in cold 100 mM formic acid (FA)/sodium formate buffer (pH 3.3) to a concentration of 6.25 U/mL, aliquoted, and stored at −80° C. until use. Active CatB (stock conc 20 ng/ul, Sigma-Aldrich, Saint Louis, MO, USA). Aqueous solutions of 0.08 mg/mL NEP (human, recombinant, amino acid 53-750, catalog number BML-SE532-0010, Enzo Life Sciences, Lausen, Switzerland), 0.55 mg/mL IDE (rat, recombinant, amino acid 43-1019, catalog number 18601-04) and 0.50 mg/mL ECE-1 (mouse, recombinant, amino acid 89-769, catalog number E3650-02) both purchased from USBiological (MA, USA) were stored at −80° C. until use. In accordance with the manufacturers' recommendation, the recombinant enzymes were not purified prior to use, and negative control experiments were not conducted. ZnCl2 (0.1 M), aqueous NH3 25% (v/v), and LC-MS quality water and acetonitrile containing 0.1% FA was obtained from Sigma-Aldrich. For buffer preparation and pH adjustment, NaCl, HCl and MES hemisodium salt was purchased from Sigma-Aldrich while NaOH and tris(hydroxymethyl)aminomethane (tris) was obtained from Merck (Darmstadt, Germany).

Enzymatic Digestion

α-Synuclein, Tau and MBP, all at a final concentration of 30 ng/ul were digested with each of the enzymes to determine the peptide degradation pattern. The enzyme to substrate ratio was 1:50 for CatB and CatD, and 1:25 for IDE, ECE-1, and NEP. A MES buffer (50 mM MES, 0.1 M NaCl, pH 6.0) was used for ECE-1 hydrolysis, A MES buffer (25 mM MES, 0.05 M NaCl, pH 5.0) was used for CatB hydrolysis, a tris buffer (50 mM tris-HCl, 0.1 M NaCl, pH 7.5) for IDE, a tris buffer (50 mM tris, pH 9.0) for NEP, and Cat D digestion was performed in a 100 mM sodium formate/FA buffer (pH 3.3), all according to manufacturer's recommendations. The incubations were conducted at 37° C. for various times (0.5, 10, 30, 60, 90 120, 150, 180 and 210 min). ZnCl2 was added to IDE and ECE-1 digestions (54 and 73 pmol, respectively) to ensure high enzyme activity. After the desired time, the enzymatic reactions were quenched by the addition of aqueous NH3 (25% v/v) ammonium hydroxide solution for CatD and concentrated FA for CatB, ECE-1, IDE and NEP to a final concentration of 1% (v/v).

Liquid Chromatography-Mass Spectrometry

All LC-MS equipment, including the columns, was obtained from Thermo Scientific (Waltham, MA, USA). The LC separation was performed with an Accela 1250 UHPLC pump and a PAL autosampler connected with a C4 Accucore column (2.1 mm inner diameter (i.d.)×150 mm length (L), 2.6 μm particles) for peptides obtained from IDE, ECE-1 and NEP proteolysis, whereas a BioBasic C18 (2.1 mm i.d.×50 mm L, 5 μm particles) was used for peptides proteolyzed with Cat D. A flow rate of 300 μL/min was used for all analyses and injection volume was set to 20 μL. A solvent gradient running from 1 to 45% acetonitrile in water with 0.1% formic acid (FA) in 20 min was applied for peptide separation on the Accucore column, and a methanol gradient running from 2 to 70% methanol water (0.1% FA) in 27.5 min was used for the BioBasic column. Solvent was diverted to waste for 0.5 and 2.5 min for the BioBasic and Accucore columns, respectively.

Mass spectrometry was performed on a Q-Exactive Orbitrap with a heated electrospray ionization source, operated at +3.2 kV. Data were recorded by data-dependent MS/MS, using a resolution of 70,000 in MS and 17,500 in MS/MS, a scan range from m/z 400 to 1500, and an AGC target of 3e6 (MS) and 2e5 (MS/MS). Normalized collision energy was set from 25 to 35% and underfill ratio was set to 1%. A dynamic exclusion of 30 s and exclusion of peptides with charge of +1 and ≥+6 was applied. Monoprotonated peptides (charge of +1) were analyzed separately using a targeted MS/MS method for positive identification.

Results

The results are shown in FIGS. 11-14. In particular, these Figures show that the enzymes target a number of cut sites, but also that each protein includes at least one region which has a low number of, or no, cut sites. In particular, for α-synuclein, these regions resistant to degradation are residues 39-53, 53-72 and 76-90; for Tau, the region resistant to degradation is residues 281-322; and for MBP the region resistant to degradation is residues 30-70. These results show that it can be expected that these degradation-resistant fragments are retained in MPS cells and can therefore be detected.

1. An ex vivo method of determining the effect of an agent on catalysis by a Monocyte Phagocyte System (MPS) cell of a disease marker product and/or at least one fragment thereof associated with neurodegeneration and/or inflammatory activation, wherein said method comprises:
i) maintaining a sample of MPS cells under conditions in which the MPS cells remain alive,
ii) exposing the sample of MPS cells to an agent and a disease marker product, to permit phagocytosis of the disease marker product by the MPS cells,
iii) detecting the intracellular amount of the disease marker product and/or at least one fragment thereof in the sample of MPS cells, and
iv) comparing the intracellular amount of the disease marker product and/or the at least one fragment thereof to an intracellular amount of the same disease marker product and/or at least one fragment thereof measured in control MPS cells in the absence of the agent,
wherein the effect of the agent on catalysis by MPS cells of the disease marker product and/or at least one fragment thereof is determined by the result of the comparison of step iv), and wherein, when the disease marker product is Aβ₁₋₄₂ (SEQ ID NO: 1) or Aβ₁₋₄₀ (SEQ ID NO: 2), step iii) comprises detecting the intracellular amount of a fragment of Aβ₁₋₄₀ (SEQ ID NO: 2) or a fragment of Aβ₁₋₄₂ (SEQ ID NO: 1) comprising residue 41 of Aβ₁₋₄₂ (SEQ ID NO: 1) by detecting a region of the fragment comprising at least 4 amino acids including residue 21, residue 34 or residue 40 of Aβ₁₋₄₂ (SEQ ID NO: 1) or Aβ₁₋₄₀ (SEQ ID NO: 2).

2. A method according to claim 1, wherein the MPS cells are microglial cells, macrophages or monocytes, preferably monocytes.

3. A method according to claim 2, wherein the MPS cells are monocytes and the method further comprises differentiating the monocytes into macrophages.

4. A method according to claim 1, wherein the MPS cells are obtained from a human subject.

5. A method according to claim 4, wherein the MPS cells are obtained from a CSF sample or a blood sample, preferably from a blood sample.

6. A method according to claim 1, wherein step ii) comprises culturing the MPS cells.

7. A method according to claim 1, wherein the agent is a stimulator or inhibitor of the MPS autophagic and/or endolysosomal systems, or is an inflammatory activator, preferably wherein the agent is Aβ₁₋₄₂, Aβ₁₋₄₀, a FFAR4 agonist, an α7 nAChR agonist or PAM, Rapamycin, Bafilomycin, Neprilysin inhibitor, IDE inhibitor, BACE inhibitor, a combination of a FFAR4 agonist and an α7 nAChR agonist or PAM, or a combination of IDE inhibitor, Neprilysin inhibitor and BACE inhibitor, preferably wherein the FFAR4 agonist is DHA, the α7 nAChR agonist or PAM is NS1738, and the combination of a FFAR4 agonist and an α7 nAChR agonist or PAM is combination of DHA and NS1738.

8. A method according to claim 1, wherein the disease marker product and/or at least one fragment thereof is Tau protein, α-synuclein, myelin basic protein (MBP) or TDP-43, or a fragment thereof, or the fragment is a fragment of Aβ₁₋₄₀ (SEQ ID NO: 2) or a fragment of Aβ₁₋₄₂ (SEQ ID NO: 1) comprising residue 41 of Aβ₁₋₄₂ (SEQ ID NO: 1).

9. A method according to claim 1, wherein the disease marker product and/or at least one fragment thereof is associated with Alzheimer's disease.

10. (canceled)

11. A method according to claim 1, wherein step iii) comprises detecting the fragment of Aβ₁₋₄₂ (SEQ ID NO: 1) or Aβ₁₋₄₀ (SEQ ID NO: 2) by detecting a region of the fragment comprising at least 4 amino acids comprising residues 21-24, residues 31-34 or residues 39-40 of Aβ₁₋₄₂ (SEQ ID NO: 1) or Aβ₁₋₄₀ (SEQ ID NO: 2).

12. A method according to claim 1, wherein step iii) comprises detecting the fragment of Aβ₁₋₄₂ (SEQ ID NO: 1) or Aβ₁₋₄₀ (SEQ ID NO: 2) by detecting the N-terminal of SEQ ID NO: 6, the C-terminal of SEQ ID NO: 6, a region of the fragment comprising residues 21-29 of Aβ₁₋₄₂ (SEQ ID NO: 1) or Aβ₁₋₄₀ (SEQ ID NO: 2), and/or a region of the fragment comprising residues 34-40 or 34-41 of API-42 (SEQ ID NO: 1) or Aβ₁₋₄₀ (SEQ ID NO: 2).

13. A method according to claim 1, wherein step iii) comprises detecting the fragment of Aβ₁₋₄₂ (SEQ ID NO: 1) or Aβ₁₋₄₀ (SEQ ID NO: 2) by detecting more than one of the N-terminal or C-terminal of SEQ ID NO: 6 and the regions of Aβ₁₋₄₂ (SEQ ID NO: 1) or Aβ₁₋₄₀ (SEQ ID NO: 2), preferably by detecting
i) a region of the fragment comprising at least 4 amino acids comprising residues 21-24 and a region of the fragment comprising at least 4 amino acids comprising residues 31-34 or 39-40,
ii) the N-terminal of SEQ ID NO: 6 and a region of the fragment comprising residues 34-40 or 34-41 of Aβ₁₋₄₂ (SEQ ID NO: 1) or Aβ₁₋₄₀ (SEQ ID NO: 2), preferably residues 1-6 of SEQ ID NO: 6 and a region of the fragment comprising residues 34-40 or 34-41 of Api-42 (SEQ ID NO: 1) or Aβ₁₋₄₀ (SEQ ID NO: 2);
iii) the C-terminal of SEQ ID NO: 6 and a region of the fragment comprising residues 21-24, preferably 21-29, of Aβ₁₋₄₂ (SEQ ID NO: 1) or Aβ₁₋₄₀ (SEQ ID NO: 2); and/or
iv) a region of the fragment comprising residues 21-24, preferably residues 21-29, of Ai-42 (SEQ ID NO: 1) or Aβ₁₋₄₀ (SEQ ID NO: 2) and a region of the fragment comprising residues 34-40 or 34-41 of Aβ₁₋₄₂ (SEQ ID NO: 1) or Aβ₁₋₄₀ (SEQ ID NO: 2).

14. A method according to claim 1, wherein the at least one fragment is Aβ₁₋₄₂ (SEQ ID NO: 1) or Aβ₁₋₄₀ (SEQ ID NO: 2) which has cleaved between amino acid residues 19 and 20 and/or between amino acid residues 34 and 35.

15. A method according to claim 1, wherein step iii) of the method comprises using antibodies to detect the fragments of Aβ₁₋₄₂ (SEQ ID NO: 1) or Aβ₁₋₄₀ (SEQ ID NO: 2), preferably an antibody specific for SEQ ID NO: 3 or 7, and an antibody specific for the C-terminal of SEQ ID NO: 6 or specific for SEQ ID NO: 4, and preferably also an antibody specific for SEQ ID NO: 5, 8 or 9.

16. A method according to claim 1, wherein step iii) comprises detecting the intracellular amount of at least one fragment of Tau, α-synuclein, TDP-43 or MBP.

17. A method according to claim 16, wherein the fragment is selected from:
Tau which has been cleaved between amino acid residues 280 and 281 and/or between amino acid residues 322 and 323; or between amino acid residues 305 and 306 and/or amino acid residues 322 and 323;
α-synuclein which has been cleaved between amino acid residues 38 and 39 and/or between amino residues 53 and 54; between amino acid residues 52 and 53 and/or amino acid residues 72 and 73; or between amino acid residues 75 and 76 and/or between amino acid residues 90 and 91;
TDP-43 which has been cleaved between amino acid residues 310 and 311 and/or between amino acid residues 334 and 335 or between amino acid residues 320 and 321 or amino acid residues 323 and 324 or amino acid residues 360 and 261; between amino acid residues 245 and 246 and/or between amino acid residues 255 and 256; or between amino acid residues 285 and 285 and/or amino acid residues 331 and 332; and
MBP which has been cleaved between amino acid residues 29 and 30 and/or between residues 70 and 71.

18. A method according to claim 17, wherein the fragment is selected from:
Tau protein which has been cleaved between amino acid residues 280 and 281 and/or between amino acid residues 322 and 323;
α-synuclein which has been cleaved between amino acid residues 38 and 39 and/or between amino acid residues 53 and 54;
TDP-43 which has been cleaved between amino acid residues 310 and 311 and/or between amino acid residues 334 and 335; and
MBP which has been cleaved between amino acid residues 29 and 30 and/or between residues 70 and 71.

19. A method according to claim 1, wherein step iii) comprises detecting the intracellular amount of more than one fragment of the disease marker product.

20. A method according to claim 1, wherein, in step ii), the agent is a stimulator or activator of the MPS autophagic and/or endolysosomal systems, and step ii) comprises exposing the sample of MPS cells to an inflammatory activator in addition to the agent, preferably wherein the inflammatory activator is LPS.

21. A method according to claim 1, wherein the control MPS cells are cells which have undergone steps i) and ii) of claim 1, except that the control MPS cells have not been exposed to the agent.

22. A method according to claim 1, wherein the method is for comparing the effects of a panel of agents on catalysis of the disease marker product and/or at least one fragment thereof, and identifying the agent with the greatest effect on catalysis of the disease marker product and/or at least one fragment thereof, or
the method is for screening agents for use in the treatment of the neurodegeneration and/or inflammatory activation and/or a condition associated with neurodegeneration, or
the method is for use in diagnosing neurodegeneration and/or inflammatory activation and/or a disease or condition associated with neurodegeneration, or
the method is for use in personalised medicine, for determining whether an agent is an effective treatment for a human subject, wherein step i) of the method comprises maintaining a sample of MPS cells obtained from a human subject under conditions in which the MPS cells remain alive, and wherein the effectiveness of the agent on the human subject is determined by the result of the comparison of step iv).

23-25. (canceled)

26. A kit comprising:
one or more antibodies selected from an antibody specific for a region of a fragment of Aβ₁₋₄₂ (SEQ ID NO: 1) or Aβ₁₋₄₀ (SEQ ID NO: 2) comprising at least 4 amino acids comprising residue 21 of Aβ₁₋₄₂ (SEQ ID NO: 1) or Aβ₁₋₄₀ (SEQ ID NO: 2), an antibody specific for a fragment of Aβ₁₋₄₂ (SEQ ID NO: 1) or Aβ₁₋₄₀ (SEQ ID NO: 2) comprising at least 4 amino acids comprising residue 34 of Aβ₁₋₄₂ (SEQ ID NO: 1) or Aβ₁₋₄₀ (SEQ ID NO: 2) and an antibody specific for a fragment of Aβ₁₋₄₂ (SEQ ID NO: 1) or Aβ₁₋₄₀ (SEQ ID NO: 2) comprising at least 4 amino acids comprising residue 40 of Aβ₁₋₄₂ (SEQ ID NO: 1) or Aβ₁₋₄₀ (SEQ ID NO: 2);
one or more antibodies selected from an antibody specific for a region of a fragment of α-synuclein (SEQ ID NO: 10) comprising at least 4 amino acids comprising residue 39 of α-synuclein (SEQ ID NO: 10), an antibody specific for a region of a fragment of α-synuclein (SEQ ID NO: 10) comprising at least 4 amino acids comprising residue 53 of α-synuclein (SEQ ID NO: 10), an antibody specific for a region of a fragment of α-synuclein (SEQ ID NO: 10) comprising at least 4 amino acids comprising residue 72 of α-synuclein (SEQ ID NO: 10), an antibody specific for a region of a fragment of α-synuclein (SEQ ID NO: 10) comprising at least 4 amino acids comprising residue 76 of α-synuclein (SEQ ID NO: 10) and an antibody specific for a region of a fragment of α-synuclein (SEQ ID NO: 10) comprising at least 4 amino acids comprising residue 90 of α-synuclein (SEQ ID NO: 10);
one or more antibodies selected from an antibody specific for a region of a fragment of Tau (SEQ ID NO: 14) comprising at least 4 amino acids comprising residue 306 of Tau (SEQ ID NO: 14), an antibody specific for a region of a fragment of Tau (SEQ ID NO: 14) comprising at least 4 amino acids comprising residue 281 of Tau (SEQ ID NO: 14) and an antibody specific for a region of a fragment of Tau (SEQ ID NO: 14) comprising at least 4 amino acids comprising residue 322 of Tau (SEQ ID NO: 14);
one or more antibodies selected from an antibody specific for a region of a fragment of TDP-43 (SEQ ID NO: 17) comprising at least 4 amino acids comprising residue 311 of TDP-43 (SEQ ID NO: 17), an antibody specific for a region of a fragment of TDP-43 (SEQ ID NO: 17) comprising at least 4 amino acids comprising residue 334 of TDP-43 (SEQ ID NO: 17), an antibody specific for a region of a fragment of TDP-43 (SEQ ID NO: 17) comprising at least 4 amino acids comprising residue 246 of TDP-43 (SEQ ID NO: 17) and an antibody specific for a region of a fragment of TDP-43 (SEQ ID NO: 17) comprising at least 4 amino acids comprising residue 255 of TDP-43 (SEQ ID NO: 17); or
one or more antibodies selected from an antibody specific for a region of a fragment of MBP (SEQ ID NO: 20) comprising at least 4 amino acids comprising residue 30 of MBP (SEQ ID NO: 20) and an antibody specific for a region of a fragment of MBP (SEQ ID NO: 20) comprising at least 4 amino acids comprising residue 70 of MBP (SEQ ID NO: 20).

27. A kit according to claim 26, wherein the kit comprises:
an antibody specific for a region of a fragment of Aβ₁₋₄₂ (SEQ ID NO: 1) or Aβ₁₋₄₀ (SEQ ID NO: 2) comprising at least 4 amino acids comprising residue 21 of Aβ₁₋₄₂ (SEQ ID NO: 1) or Aβ₁₋₄₀ (SEQ ID NO: 2), an antibody specific for a fragment of Aβ₁₋₄₂ (SEQ ID NO: 1) or Aβ₁₋₄₀ (SEQ ID NO: 2) comprising at least 4 amino acids comprising residue 34 of Aβ₁₋₄₂ (SEQ ID NO:
1) or Aβ₁₋₄₀ (SEQ ID NO: 2) and an antibody specific for a fragment of Aβ₁₋₄₂ (SEQ ID NO: 1) or Aβ₁₋₄₀ (SEQ ID NO: 2) comprising at least 4 amino acids comprising residue 40 of Aβ₁₋₄₂ (SEQ ID NO: 1) or Aβ₁₋₄₀ (SEQ ID NO: 2);
an antibody specific for a region of a fragment of α-synuclein (SEQ ID NO: 10) comprising at least 4 amino acids comprising residue 39 of α-synuclein (SEQ ID NO: 10) and an antibody specific for a region of a fragment of α-synuclein (SEQ ID NO: 10) comprising at least 4 amino acids comprising residue 53 of α-synuclein (SEQ ID NO: 10);
an antibody specific for a region of a fragment of Tau (SEQ ID NO: 14) comprising at least 4 amino acids comprising residue 281 of Tau (SEQ ID NO: 14) and an antibody specific for a region of a fragment of Tau (SEQ ID NO: 14) comprising at least 4 amino acids comprising residue 322 of Tau (SEQ ID NO: 14);
an antibody specific for a region of a fragment of TDP-43 (SEQ ID NO: 17) comprising at least 4 amino acids comprising residue 311 of TDP-43 (SEQ ID NO: 17) and an antibody specific for a region of a fragment of TDP-43 (SEQ ID NO: 17) comprising at least 4 amino acids comprising residue 334 of TDP-43 (SEQ ID NO: 17); or
an antibody specific for a region of a fragment of MBP (SEQ ID NO: 20) comprising at least 4 amino acids comprising residue 30 of MBP (SEQ ID NO: 20) and an antibody specific for a region of a fragment of MBP (SEQ ID NO: 20) comprising at least 4 amino acids comprising residue 70 of MBP (SEQ ID NO: 20).


SEQUENCE LISTING (21 sequences)

SEQ ID NO: 1 - protein, 42 aa, Homo sapiens [sequence omitted]

SEQ ID NO: 2 - protein, 40 aa, Homo sapiens
DAEFRHDSGYEVHHQKLVFFAEDVGSNKGAIIGLMVGGVV

SEQ ID NO: 3 - protein, 6 aa, Homo sapiens
FAEDVG

SEQ ID NO: 4 - protein, 6 aa, Homo sapiens
GAIIGL

SEQ ID NO: 5 - protein, 4 aa, Homo sapiens
VVIA

SEQ ID NO: 6 - protein, 15 aa, Homo sapiens
FAEDVGSNKGAIIGL

SEQ ID NO: 7 - protein, 9 aa, Homo sapiens
AEDVGSNKG

SEQ ID NO: 8 - protein, 8 aa, Homo sapiens
LMVGGVVI

SEQ ID NO: 9 - protein, 7 aa, Homo sapiens
LMVGGVV

SEQ ID NO: 10 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 11 - protein, 15 aa, Homo sapiens
YVGSKTKEGVVHGVA

SEQ ID NO: 12 - protein, 20 aa, Homo sapiens
ATVAEKTKEQVTNVGGAVVT

SEQ ID NO: 13 - protein, 15 aa, Homo sapiens
AVAQKTVEGAGSIAA

SEQ ID NO: 14 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 15 - protein, 17 aa, Homo sapiens
VQIVYKPVDLSKVTSKC

SEQ ID NO: 16 - protein, 42 aa, Homo sapiens
KLDLSNVQSKCGSKDNIKHVPGGGSVQIVYKPVDLSKVTSKC

SEQ ID NO: 17 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 18 - protein, 24 aa, Homo sapiens
MNFGAFSINPAMMAAAQAALQSSW

SEQ ID NO: 19 - protein, 10 aa, Homo sapiens
EDLIIKGISV

SEQ ID NO: 20 - protein, 220 aa, Homo sapiens
MGSSHHHHHHSSGLVPRGSHMGSMGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKVSSEE

SEQ ID NO: 21 - protein, 41 aa, Homo sapiens
KRELNAEKASTNSETNRGESEKKRNLGELSRTTSEDNEVFG